(19) United States
(12) Patent Application Publication
(10) Pub. No.: US 2024/0285286 A1
(43) Pub. Date: Aug. 29, 2024

(54) DEVICES AND METHODS FOR APPLYING A HEMOSTATIC CLIP ASSEMBLY

(71) Applicant: Conmed Corporation, Largo, FL (US)

(72) Inventors: Michael Barenboym, Boston, MA (US); Daniel P. Damato, San Diego, CA (US); Tori Alexander, Auburn, MA (US); Sabaris Chinnar Suresh Kumar, Peoria, IL (US)

(73) Assignee: Conmed Corporation, Largo, FL (US)

(21) Appl. No.: 18/568,216

(22) PCT Filed: Apr. 13, 2022
(86) PCT No.: PCT/US2022/024654

§ 371 Date: Dec. 7, 2023

Related U.S. Application Data
(60) Provisional application No. 63/208,523, filed on Jun. 9, 2021.

(51) Int. Cl. A61B 17/128; A61B 17/122

(52) U.S. Cl. CPC A61B 17/1285; A61B 17/122

(57) ABSTRACT

The device includes a proximal delivery catheter having a proximal handle assembly and an elongated catheter body extending distally from the proximal handle assembly. The elongated catheter body defines a longitudinal axis. The proximal delivery catheter includes a drive wire movably positioned within the elongated catheter body, a spring release coupled to a distal end of the drive wire, and a shaft spring positioned radially outward from the spring release. The shaft spring includes an annular portion. The spring release is configured and adapted to abut the annular portion of the shaft spring upon proximal translation of the spring release. The device includes a distal clip assembly removably connected to the distal end of the elongated catheter body. The proximal delivery catheter is configured and adapted to transmit linear motion along the longitudinal axis and torsion about the longitudinal axis to at least a portion of the distal clip assembly.

### CROSS-REFERENCE TO RELATED APPLICATIONS

This application claims the benefit of priority to U.S. Provisional Patent Application Ser. No. 63/208,523, filed Jun. 9, 2021, the disclosure of which is herein incorporated by reference in their entirety.

### BACKGROUND OF THE INVENTION

### 1. Field of the Invention

The subject invention is directed to surgical equipment, and more particularly to hemostatic clips used endoscopic surgical procedures.

### 2. Description of Related Art

Endoscopic or “minimally invasive” hemostatic clips are used in performance of hemostasis to stop and prevent re-bleeding, or in procedures such as ampullectomy, polypectomy, tissue repair and correction of other tissue defects. Such procedures are typically performed by grasping the tissue with the hemostatic clip. Benefits of using hemostatic clips in such procedures include reduced trauma to the patient, low re-bleeding rate, reduced opportunity for infection, and decreased recovery time. Some applications for hemostasis clips include closure of post resection submucosal defects, naturally occurring defects of the GI tract, and closure of luminal perforations.

The subject invention provides an improved mechanism for a hemostatic clip. The novel design allows for a shorter deployed clip body, improved tissue grasping and clip locking, and an improved disconnecting feature, which are described in detail herein below, along with other novel devices and systems.

### SUMMARY OF THE DISCLOSURE

The subject invention is directed to a new and useful surgical device for applying a hemostatic clip assembly. The device includes a proximal delivery catheter having a proximal handle assembly and an elongated catheter body extending distally from the proximal handle assembly. The elongated catheter body defines a longitudinal axis. The proximal delivery catheter includes a drive wire movably positioned within the elongated catheter body, a spring release coupled to a distal end of the drive wire, and a shaft spring positioned radially outward from the spring release. The shaft spring includes an annular portion, wherein the spring release is configured and adapted to abut the annular portion of the shaft spring upon proximal translation of the spring release. The device includes a distal clip assembly removably connected to the distal end of the elongated catheter body. The proximal delivery catheter is configured and adapted to transmit linear motion along the longitudinal axis and torsion about the longitudinal axis to at least a portion of the distal clip assembly.

In some embodiments, the shaft spring is positioned radially inward from the elongated catheter body. The annular portion of the shaft spring can be positioned around the drive wire. A proximal facing surface of the spring release can be configured and adapted to interfere with the annular portion of the shaft spring upon proximal translation of the spring release. The shaft spring can be configured and adapted to translate proximally relative to the elongated catheter body.

It is contemplated that in some embodiments the distal clip assembly includes a distal clip housing. The shaft spring can include at least one arm removably coupled to the distal clip housing. The at least one arm can include an outwardly extending flange that removably engages with an aperture defined in a proximal end of the distal clip housing. The outwardly extending flange of the at least one arm can be configured and adapted to bend and release from the aperture of the distal clip housing as the spring release moves proximally to move the shaft spring proximally relative to the distal clip housing.

The distal clip assembly can include a jaw adapter yoke slidably positioned within the distal clip assembly, and a jaw assembly having a pair of cooperating jaw members fixed to the jaw adapter yoke by a first pin. The first pin can be oriented orthogonally relative to the longitudinal axis. At least one of the jaw members can be configured and adapted to rotate about the first pin and to rotate about the longitudinal axis. The distal clip assembly can include a second pin connecting between the jaw members and the distal clip housing. Each jaw member can include a proximal body portion and a distal end effector. The proximal body portion of each jaw member can include a respective cam slot configured and adapted to receive the second pin and a pivot aperture configured and adapted to receive the first pin.

The cam slots can be configured and adapted to translate along the second pin to move axially relative to the distal clip housing and to move the jaw members between a open configuration, where respective distal tips of the jaw members are moved away from one another, a closed configuration where the respective distal tips of the jaw members are approximated towards one another to grasp tissue, and a locked configuration. Each cam slot can include a distal locking neck projecting into the cam slot defining a distal locking area. The jaw members can be in the locked configuration when the second pin is distal relative to the distal locking neck in the distal locking area.

In some embodiments, the jaw adapter yoke includes a proximal receiving portion and the spring release includes a distal portion configured and adapted to be received within the proximal receiving portion of the jaw adapter yoke to transmit axial and rotational force from the drive wire to the jaw adapter yoke. The drive wire can be coupled to a proximal portion of the spring release to transmit linear and rotational motion from the drive wire to the jaw adapter yoke. The distal portion of the spring release can be divided into at least two prongs. Each prong can have a mating surface selectively engageable with an inner surface of the receiving portion of the jaw adapter yoke. Each prong can be configured and adapted to deflect inwardly and release from the receiving portion when an axial force in a proximal direction is applied to the spring release. The jaw adapter yoke can include a pair of axially extending spaced apart arms. Each arm can include an elongated opening. The first pin can be slidably received within each elongated opening. The elongated opening can be configured and adapted to allow a first of the jaw members to be angled at a first angle relative to the longitudinal axis and a second of the jaw members to be angled at a second angle relative to the longitudinal axis.

The spring release can include a distal portion, a proximal portion, and a neck portion therebetween. The distal portion of the spring release can be configured and adapted to be received within a bore of the jaw adapter yoke to transmit axial and rotational force from the drive wire to the jaw adapter yoke.

In accordance with another aspect, a device for applying a hemostatic clip assembly includes a proximal delivery catheter including a proximal handle assembly and an elongated catheter body extending distally from the proximal handle assembly. The elongated catheter body defining a longitudinal axis, a drive wire movably positioned within the elongated catheter body. The proximal delivery catheter includes a spring release coupled to a distal end of the drive wire, and a shaft spring positioned radially outward from the spring release. The device for applying a hemostatic clip assembly includes a distal clip assembly removably connected to a distal end of the elongated catheter body. The distal clip assembly includes a distal clip housing having a distally facing retaining surface, a jaw adapter yoke slidably positioned within the distal clip assembly, and a jaw assembly having a pair of cooperating jaw members fixed to the jaw adapter yoke. The proximal delivery catheter is configured and adapted to transmit linear motion along the longitudinal axis and torsion about the longitudinal axis to at least a portion of the distal clip assembly. The shaft spring includes at least one arm removably coupled to the distally facing retaining surface of the distal clip housing.

Other aspects of the proximal delivery catheter and the distal clip assembly can be similar to those already described above. The components of spring release and shaft spring can be similar to those described above. At least one arm of the shaft spring can include an outwardly extending flange that removably engages with a distally facing retaining surface of an aperture defined in a proximal end of the distal clip housing.

In accordance with another aspect, a device for applying a hemostatic clip assembly includes a proximal delivery catheter and a distal clip assembly. The proximal delivery catheter includes a proximal handle assembly, and an elongated catheter body extending distally from the proximal handle assembly. The elongated catheter body defines a longitudinal axis. The distal clip assembly is removably connected to a distal end of the elongated catheter body. The distal clip assembly including a distal clip housing having a distally facing retaining surface. The distal clip assembly includes a jaw adapter yoke slidably positioned within the distal clip assembly, and a jaw assembly having a pair of cooperating jaw members fixed to the jaw adapter yoke with a first pin. The proximal delivery catheter is configured and adapted to transmit linear motion along the longitudinal axis and torsion about the longitudinal axis to at least a portion of the distal clip assembly. The jaw adapter yoke includes a pair of axially extending spaced apart arms. Each arm includes an elongated opening. The first pin is slidably received within each elongated opening. The elongated opening is configured and adapted to allow a first of the jaw members to be angled at a first angle relative to the longitudinal axis and a second of the jaw members to be angled at a second angle relative to the longitudinal axis. Other aspects of the proximal delivery catheter and the distal clip assembly can be similar to those already described above.

In accordance with another aspect, a hemostatic clip assembly includes a distal clip housing defining a longitudinal axis, a jaw adapter yoke slidably positioned within the distal clip housing, and a jaw assembly having a pair of cooperating jaw members fixed to the jaw adapter yoke by a first pin. The first pin is oriented orthogonally relative to the longitudinal axis. The jaw adapter yoke is configured and adapted to translate axially along the longitudinal axis and rotate about the longitudinal axis. At least one of the jaw members is configured and adapted to rotate about the first pin and to rotate about the longitudinal axis. The jaw adapter yoke includes a pair of axially extending spaced apart arms. Each arm includes an elongated opening. The first pin is slidably received within each elongated opening. The elongated opening is configured and adapted to allow a first of the jaw members to be angled at a first angle relative to the longitudinal axis and a second of the jaw members to be angled at a second angle relative to the longitudinal axis. The hemostatic clip assembly can include similar components and aspects to those described above.

In accordance with another aspect, a method for firing a hemostatic clip assembly includes positioning a distal clip assembly proximate to a target location, and translating an actuation portion of a proximal handle assembly of a proximal delivery catheter relative to a grasping portion of the proximal handle assembly in at least one of a proximal direction or a distal direction. The distal clip assembly includes a distal clip housing, a jaw adapter yoke slidably positioned within the distal clip assembly, and a jaw assembly having a pair of cooperating jaw members fixed to the jaw adapter yoke by a first pin. The proximal delivery catheter includes an elongated catheter body extending distally from the proximal handle assembly, the elongated catheter body defining a longitudinal axis, the actuation portion operatively connected to the jaw adapter yoke via a drive wire and a spring release to transmit linear motion along the longitudinal axis and torsion about the longitudinal axis to the jaw adapter yoke. The linear motion of the jaw adapter yoke transmits a linear component of motion to at least one jaw member and a cam slot of at least one jaw member to translate the cam slot along a second pin connecting between at least one of the jaw members and the distal clip housing, thereby rotating at least one of the jaw members about the first pin and to rotate about the longitudinal axis. Translating the actuation portion includes translating the spring release in the proximal direction causing abutting between the spring release and an annular portion of the shaft spring. The shaft spring is coupled to a proximal end of the distal clip housing via an outwardly extending flange of at least one arm extending from the annular portion of the shaft spring. The abutting causes the outwardly extending flange to bend inward and disengage from the proximal end of the distal clip housing.

In some embodiments, translating the actuation portion includes translating the actuation portion further in the proximal direction to transmit further linear motion in the proximal direction to the spring release. The further linear motion in a proximal direction can de-couple a distal portion of the spring release from a receiving portion of the jaw adapter yoke.

These and other features of a surgical device for applying a hemostatic clip assembly in accordance with the subject invention will become more readily apparent to those having ordinary skill in the art to which the subject invention appertains from the detailed description of the embodiments taken in conjunction with the following brief description of the drawings.

### BRIEF DESCRIPTION OF THE DRAWINGS

So that those skilled in the art will readily understand how to make and use the gas circulation system of the subject invention without undue experimentation, preferred embodiments thereof will be described in detail herein below with reference to the figures wherein:

FIG. 1 is a perspective view from the proximal direction of a device for applying a hemostatic clip assembly constructed in accordance with an embodiment of the present disclosure, showing a proximal delivery catheter having a proximal handle assembly and an elongated catheter body and the distal clip assembly;

FIG. 2 is a perspective view of the distal clip assembly of FIG. 1, showing a jaw assembly with a pair of cooperating jaw members operatively connected to the distal clip housing;

FIG. 3 is an exploded perspective view of a portion of the device of FIG. 1, showing the showing the distal end of the proximal delivery catheter and the distal clip assembly;

FIG. 4 is a cross-sectional perspective view of the distal clip housing of FIG. 1, showing a distal facing stop surface;

FIG. 5 is a side elevation view of the jaw member of FIG. 1, showing proximal and distal portions of the cam slot;

FIG. 6 is a perspective cross-sectional view of a jaw adapter yoke of the device of FIG. 1 from a distal direction, showing an elongated pin aperture of the jaw adapter yoke;

FIG. 7 is a cross-sectional side view of the spring release of FIG. 1, showing the mating surfaces of the prongs of the distal portion of the spring release;

FIG. 8 is a perspective view of a shaft spring of the device of FIG. 1 from a proximal direction, showing an annular portion of the shaft spring;

FIG. 9A is a cross-sectional side elevation view of a portion of the device of FIG. 1, showing the jaw members in the open configuration where respective distal tips of the jaw members are moved away from one another to grasp a target area of tissue;

FIG. 9B is a cross-sectional side elevation view of a portion of the device of FIG. 1, showing the jaw members in the open configuration in a proximally canted position;

FIG. 9C is a cross-sectional side elevation view of a portion of the device of FIG. 1, showing the jaw members in the open configuration in a distally canted position;

FIG. 10 is a cross-sectional side elevation view of a portion of the device of FIG. 1, showing the jaw members in a partially closed configuration where the respective distal tips of the jaw members are approximated towards one another to grasp tissue;

FIG. 11 is a cross-sectional side elevation view of a portion of the device of FIG. 1, showing the jaw members in a closed configuration where the respective distal tips of the jaw members are approximated towards one another to grasp tissue;

FIG. 12A is a cross-sectional side elevation view of a portion of the device of FIG. 1, showing the transition of the second pin to a locked position within the cam slot;

FIG. 12B is a cross-sectional side elevation detail view of a portion of the device of FIG. 1, schematically showing the transition of the second pin to a locked position within the cam slot;

FIG. 13 is a cross-sectional side elevation view of a portion of the device of FIG. 1, showing the spring release engaged with the jaw adapter yoke in the closed, locked configuration and schematically showing the inward deflection of the prongs of the spring release;

FIG. 14 is a cross-sectional side elevation view of a portion of the device of FIG. 1, showing the release of the spring release from the jaw adapter yoke and the abutment of the spring release with the annular portion of the shaft spring;

FIG. 15 is a cross-sectional side elevation view of a portion of the device of FIG. 1, showing the outward extending flanges bending inward as the shaft spring moves proximally relative to the distal clip housing;

FIG. 16 is a cross-sectional side elevation view of a portion of the device of FIG. 1, showing the outward extending flanges being released from the apertures of the distal clip housing;

FIG. 17 is a cross-sectional side elevation view of a portion of the device of FIG. 1, showing the spring release and shaft spring disconnected from the jaw adapter yoke and distal clip housing for removal of the proximal delivery catheter;

FIG. 18 is a cross-sectional axial view of the drive wire of the device of FIG. 1, showing the filars of the drive wire;

FIG. 19 is a side view of a portion of the drive wire of the device of FIG. 1, showing the filars of the drive wire wound about a central filar;

FIG. 20 is a perspective view from the proximal direction of a device for applying a hemostatic clip assembly constructed in accordance with another embodiment of the present disclosure, showing a proximal delivery catheter having a proximal handle assembly and an elongated catheter body and the distal clip assembly;

FIG. 21 is a perspective view of the spring release of the device of FIG. 20, showing the shoulder boss on the spring release;

FIG. 22 is a cross-sectional side view of the spring release of the device of FIG. 20, showing the proximally facing surface of the shoulder boss;

FIG. 23 is a perspective view of the yoke of the device of FIG. 20 from a proximal direction, showing the proximal receiving portion;

FIG. 24 is a perspective cross-sectional view of the yoke of the device of FIG. 20, showing the inside surface of the yoke;

FIG. 25 is a cross-sectional side elevation view of a portion of the device of FIG. 20, showing the jaw members in the open configuration where respective distal tips of the jaw members are moved away from one another to grasp a target area of tissue;

FIG. 26 is a cross-sectional side elevation view of a portion of the device of FIG. 20, showing the jaw members in a partially closed configuration where the respective distal tips of the jaw members are approximated towards one another to grasp tissue;

FIG. 27 is a cross-sectional side elevation view of a portion of the device of FIG. 20, showing the jaw members in a closed configuration where the respective distal tips of the jaw members are approximated towards one another to grasp tissue;

FIG. 28 is a cross-sectional side elevation view of a portion of the device of FIG. 20, showing the transition of the second pin to a locked position within the cam slot;

FIG. 29 is a cross-sectional side elevation view of a portion of the device of FIG. 20, showing the prongs of the spring release being compressed inwardly as the spring release moves proximally relative to the yoke;

FIG. 30 is a cross-sectional side elevation view of a portion of the device of FIG. 20, showing the spring release moving proximally relative to the yoke;′

FIG. 31 is a cross-sectional side elevation view of a portion of the device of FIG. 20, showing the spring release moving proximally relative to the yoke toward the annular portion of the shaft spring;

FIG. 32 is a cross-sectional side elevation view of a portion of the device of FIG. 20, showing the shoulder boss of the spring release abutting the annular portion of the shaft spring;

FIG. 33 is a cross-sectional side elevation view of a portion of the device of FIG. 20, showing the outward extending flanges bending inward as the shaft spring moves proximally relative to the distal clip housing;

FIG. 34 is a cross-sectional side elevation view of a portion of the device of FIG. 20, showing the outward extending flanges released from the apertures of the distal clip housing as the shaft spring moves proximally relative to the distal clip housing;

FIG. 35 is a cross-sectional side elevation view of a portion of the device of FIG. 30, showing the spring release and shaft spring disconnected from the jaw adapter yoke and distal clip housing for removal of the proximal delivery catheter;

FIG. 36 is a side elevation view of the jaw member of FIG. 20, showing proximal and distal portions of the cam slot;

FIG. 37 is a side elevation view of another embodiment of a jaw member constructed in accordance with another embodiment of the present disclosure for a device for applying a hemostatic clip assembly, showing a linear triangular ramp;

FIG. 38 is a side elevation view of another embodiment of a jaw member constructed in accordance with another embodiment of the present disclosure for a device for applying a hemostatic clip assembly, showing a slot in the jaw member;

FIG. 39 is a side elevation view of another embodiment of a jaw member constructed in accordance with another embodiment of the present disclosure for a device for applying a hemostatic clip assembly, showing a reverse slope in the distal portion of the cam slot;

FIG. 40 is a top view of another embodiment of a release pin constructed in accordance with another embodiment of the present disclosure for a device for applying a hemostatic clip assembly, showing a tapered distal end of the release pin engaged with the yoke of the device of FIG. 1;

FIG. 41 is a cross-sectional side view of another embodiment of a distal clip housing constructed in accordance with another embodiment of the present disclosure for a device for applying a hemostatic clip assembly, showing a proximal end of the distal clip end being tapered radially inwardly and engaged with the shaft spring of the device of FIG. 1;

FIG. 42 is a side view of another embodiment of a distal clip housing constructed in accordance with another embodiment of the present disclosure for a device for applying a hemostatic clip assembly, showing a proximal end of the distal clip end having u-shaped slots engaged with the shaft spring of the device of FIG. 1;

FIG. 43 is a front view of a portion of the device of FIG. 1, showing the jaw members of the jaw assembly each having four pointed teeth;

FIG. 44 is a front view of another embodiment of a jaw assembly constructed in accordance with another embodiment of the present disclosure, showing each jaw member with two rounded peaks for atraumatic contact; and

FIG. 45 is a perspective view of a shaft spring of the device of FIG. 20, from a proximal direction, showing an annular portion of the shaft spring.

### DETAILED DESCRIPTION OF THE EMBODIMENTS

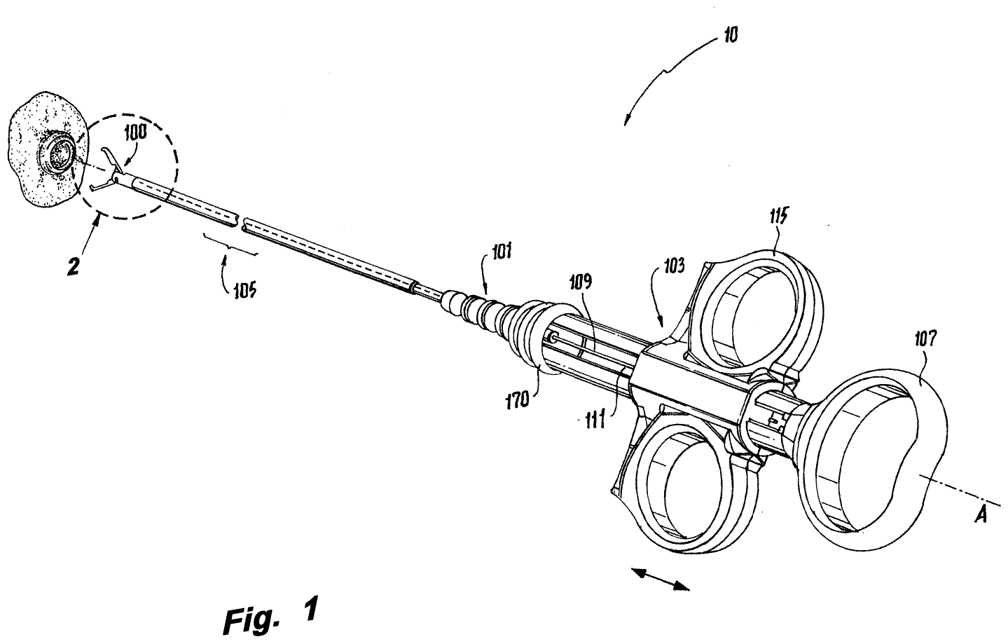

Referring now to the drawings wherein like reference numerals identify similar structural elements and features of the subject invention, there is illustrated in FIG. 1 a surgical device for applying a hemostatic clip assembly in a patient, and more particularly, for separating the hemostatic clip assembly to function as a short-term implant constructed in accordance with an embodiment of the subject disclosure and is designated generally by reference numeral 10. Other embodiments of the generator control system in accordance with the disclosure are provided as will be described.

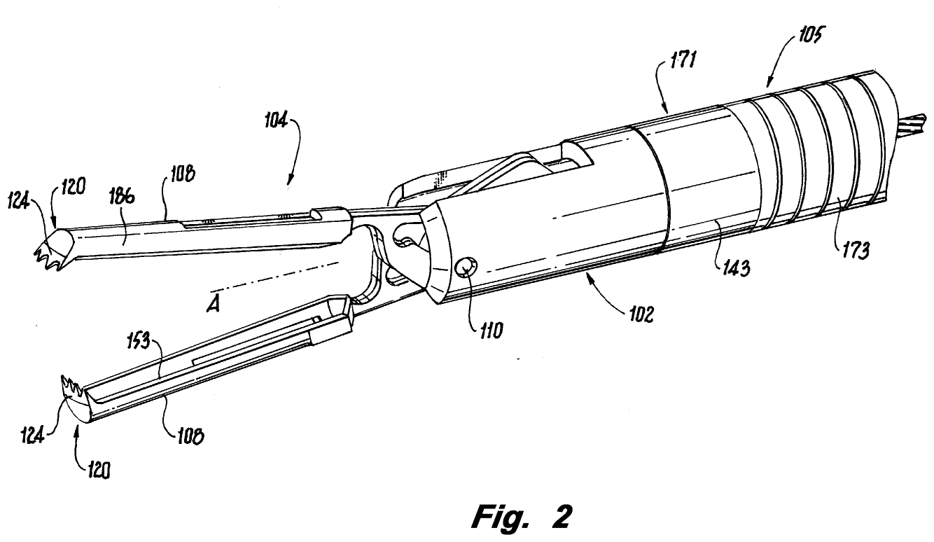

As shown in FIGS. 1-2, a surgical device 10 for applying a hemostatic clip assembly 100 includes proximal delivery catheter 101 and the distal clip assembly 100. The distal clip assembly 100, e.g., a hemostasis clip, separates from the delivery catheter 101 to function as a short-term implant to stop and prevent re-bleeding, or in procedures such as ampullectomy, polypectomy, tissue repair and correction of other tissue defects. Such procedures are typically performed by grasping the tissue with the hemostatic clip. Using hemostatic clips in such procedures can result in benefits such as reduced trauma to the patient, low re-bleeding rate, reduced opportunity for infection, and decreased recovery time.

With continued reference to FIGS. 1-2, the proximal delivery catheter 101 has a proximal handle assembly 103 and an elongated catheter body 105 extending distally from the proximal handle assembly 103. The elongated catheter body 105 defines a longitudinal axis A. The proximal handle assembly 103 includes an actuation portion 115 coupled to a proximal end 111 of the drive wire 109, and a grasping portion 107. The actuation portion 115 is configured and adapted to translate relative to the grasping portion 107 to apply an axial force to the drive wire 109. Grasping portion 107 and actuation portion 115 are configured and adapted to rotate relative to a cap 170 and catheter body 105, thereby also rotating drive wire 109. Internal annular slots on the distal portion of grasping portion 107 interact with annular tabs on inside diameter of end cap 170 to prevent axial motion of actuation portion 115 and grasping portion but allow rotation.

As shown in FIGS. 1-3, the proximal delivery catheter 101 includes a shaft spring 142 between a proximal end of the distal clip assembly 100 and a distal end 143 of the catheter body 105. The distal clip assembly 100 includes a distal clip housing 102 and a jaw assembly 104 slidably connected to the distal clip housing 102. The proximal delivery catheter 101 is configured and adapted to transmit linear motion along the longitudinal axis A and torsion about the longitudinal axis A to at least a portion of the distal clip assembly 100. The distal clip assembly 100 includes a jaw adapter yoke 106 positioned within the distal clip housing 102 and slidable relative to the distal clip housing 102. The jaw assembly 104 has a pair of cooperating jaw members 108 rotatably connected to the jaw adapter yoke 106 at a first pin 118 and slidably connected to the distal clip housing 102 by a second pin 110. The first and second pins 118 and 110, respectively, are oriented orthogonally relative to the longitudinal axis A. The first and second pins, 118 and 110, respectively, each define a respective pin rotation axis R. Both pin rotation axes R are orthogonal to the longitudinal axis A.

With reference now to FIGS. 2-3 and 6, the proximal delivery catheter 101 includes a spring release 136 having a distal portion 138 configured and adapted to be received within a proximal receiving portion 133 of the jaw adapter yoke 106. The jaw members 108 are configured and adapted to rotate about the first pin 118 between an open configuration and a closed configuration, and/or between a closed configuration and an open configuration, and to rotate about the longitudinal axis A. Jaw members 108 translate along longitudinal axis A and rotate about first pin 118 at the same time. Because first pin 118 is translating axially, the center of rotation is changing (the pin axis of first pin 118) as it shifts along the longitudinal axis A.

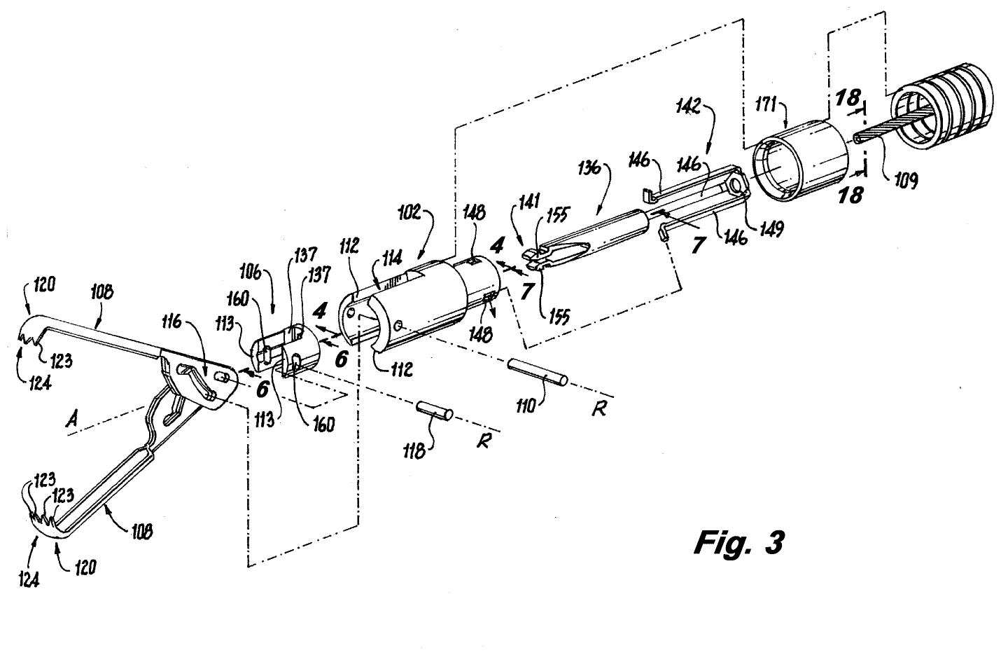

As shown in FIGS. 3 and 8, the shaft spring 142 includes arms 146 configured and adapted to be removably coupled to the distal clip housing 102, described in more detail below. Shaft spring 142 includes an annular portion 149 that defines a through-hole 150. The spring release 136 is positioned at least partially within the shaft spring 142. Annular portion 149 of the shaft spring 142 is positioned around the drive wire 109. In other words, the drive wire 109 goes through the through-hole 150. The hemostatic clip assembly 100 is removably connected to a distal end 143 of the elongated catheter body 105 via the shaft spring 142.

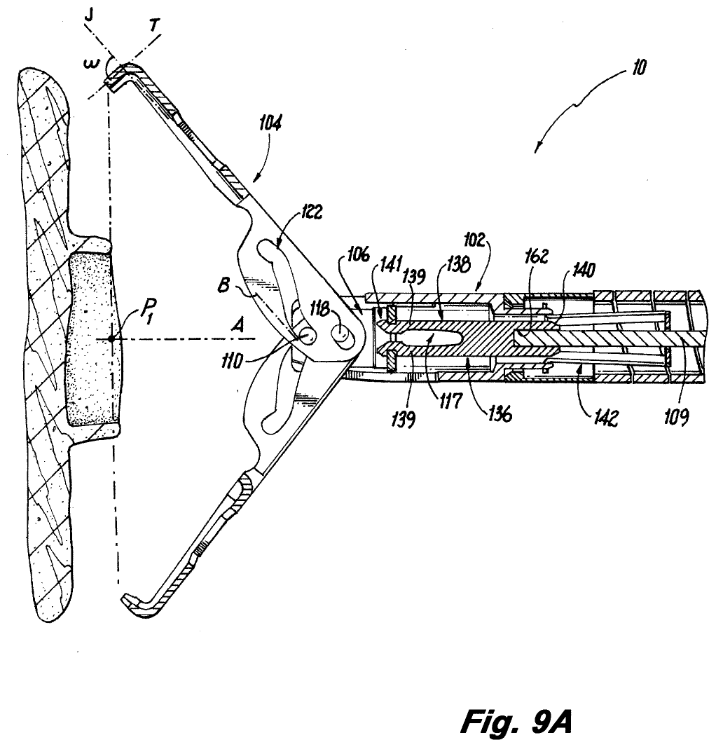

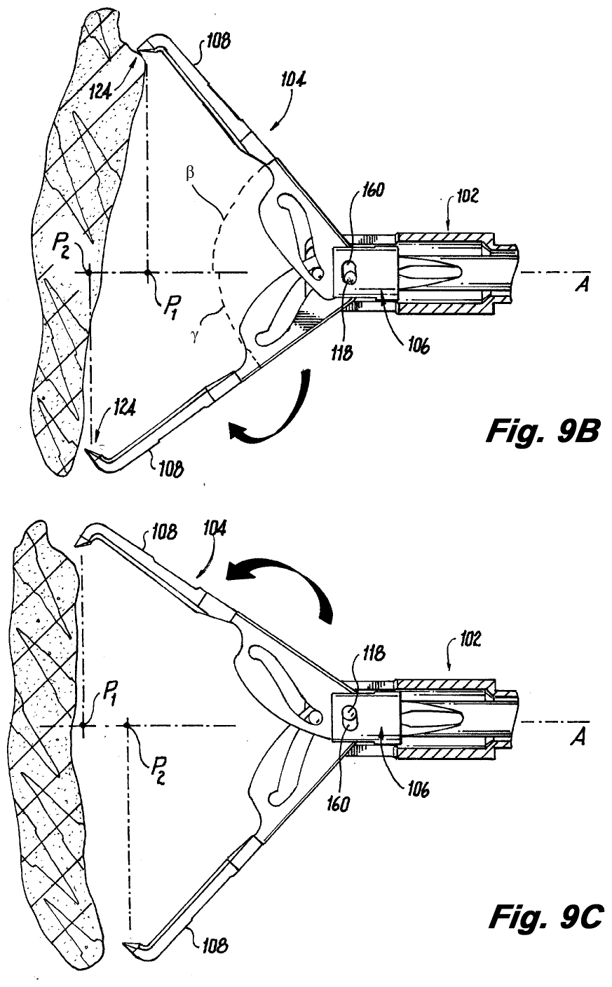

As shown in FIGS. 5 and 9A, each jaw member 108 includes a proximal body portion 116 and a distal end effector 120. The proximal body portion 116 of each jaw member 108 includes a respective cam slot 122 configured and adapted to receive the second pin 110. Jaw members 108 are driven opened and/or closed by the second pin 110, e.g., a cam pin, as cam slots 122 of the jaw members 108 slide along second pin 110. The cam slots 122 are configured adapted to slide axially along second pin 110 relative to the distal clip housing 102 to move the jaw members 108 between the open configuration where respective distal tips 124 of the jaw members 108 are moved away from one another, the closed configuration where the respective distal tips 124 of the jaw members 108 are approximated towards one another to grasp tissue, and a locked configuration.

With reference now to FIGS. 5 and 9A, each jaw member 108 includes a pivot aperture 134 configured and adapted to receive the first pin 118. Pivot aperture 134 has an elongated shape, e.g., a pill shape, where the length of aperture 134 (along longitudinal aperture axis Z) is greater than a width of the aperture (defined along lateral aperture axis Y, orthogonal to axis Z). Pivot aperture 134 is configured and adapted to increase the included angle between jaw members 108 while in the open configuration, e.g. the angle shown between distal tips 120 of jaw members 108 in FIG. 9A. Pivot aperture 134 is configured and adapted to also increase initial torque transferred to the clip arms 108 during the initiation of clip 100 closure. The distance between first pin 118 and second pin 110 as well as a comparison of a contact angle between cam slot 122 and second pin 110 with a contact angle between pivot aperture 134 and first pin 118 dictate the closing torque conversion at any given point in clip actuation. Generally, the contact angle for a given slot/aperture and its respective pin 118 or 110 can be defined as the angle between a tangent axis B and the longitudinal axis A, shown in FIG. 9A. The tangent axis B is defined at the contact point between a surface of the given slot/aperture and a surface of its corresponding pin. The ratio between respective contact angles for pin 118 and pin 110 dictates the reaction forces of each pin/contact surface interaction such that, in some embodiments, the tangent axis B between pivot aperture 134 and first pin 118 can be configured so that initial motion of first pin 118 relative to the longitudinal aperture axis Z of pivot aperture 134 occurs earlier or simultaneous to relative displacement of second pin 110 within cam slot 122. This configuration increases the effective torque translated to jaw arms 108 for a given closing force. On the other hand, the embodiment of FIGS. 9A-9C, where initial motion of first pin 118 relative to the longitudinal aperture axis Z of pivot aperture 134 occurs after relative displacement of second pin 110 within cam slot 122, can offer a larger opening angle for jaw members 108 at maximum open position, while still providing substantially equivalent torque in the closed configuration.

With continued reference to FIG. 5, in some embodiments a pivot aperture, similar to pivot aperture 134, may extend at an angle relative to longitudinal axis A in order to further customize the force response curve. In particular, the longitudinal axis Z of a pivot aperture may create an angle between zero and forty-five degrees (0-45°) in either direction relative to the longitudinal axis A. The distal end effectors 120 of each jaw member 108 include teeth 123 at their distal tips 124 that interlock with teeth of an abutting opposite jaw member. Teeth 123 positioned proximate to the side edge of their respective jaw member 108 optimize and maximize single-jaw tissue retention force during manipulation or tissue apposition, so as to catch tissue/defects easily, especially in non-perpendicular approaches to tissue. Jaw members 108 are identical mirror images of each other and can be stamped and folded over to have a 3D profile, allowing cost savings by economy of scale.

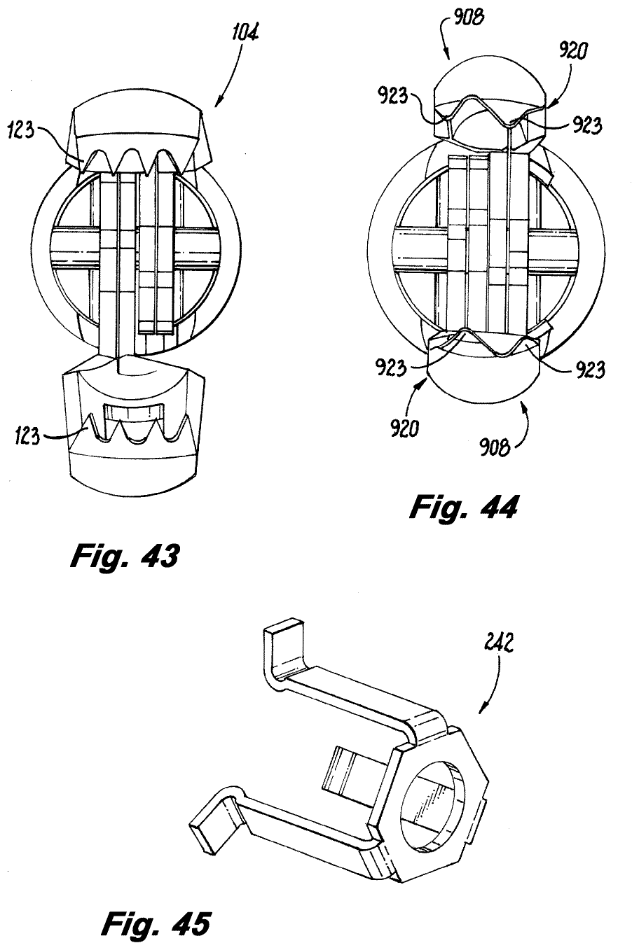

As shown in FIGS. 2-3 and 43, the end effector 120 of each jaw member 108 includes an internal concave surface 153 which also helps to maximize tissue retention and capacity. A corresponding outer convex surface 186 aids in atraumatic and smooth insertion through an endoscope working channel. Those skilled in the art will readily appreciate that distal end effectors 120 include four pointed teeth 123. Those skilled in the art will readily appreciate that a variety of end-effector styles can be used, e.g., at least one pointed tooth/peak, multiple peaks, of different or similar size. Distal end effectors 120 could also terminate in a combination of pointed peaks and rounded peaks to balance tissue pressure, allowing jaw members 108 to hook tissue with at least one peak and provide atraumatic contact with at least one peak. As shown in FIG. 44, another embodiment of a jaw member 908 can be used in device 10 in lieu of one or more of jaw members 108. Jaw member 908 includes a distal end effector 920 with all rounded peaks 923 for atraumatic contact.

As shown in FIG. 9A, the tooth (or teeth, peaks, etc.) may create an angle ω relative to an axis J of their respective jaw arms 108 between zero and 180 degrees, optimizing the approach angle of distal tips 124 relative to tissue surface. In the embodiment of FIG. 9A, the angle ω of a tip axis T relative to axis J is approximately 90 degrees. It is contemplated, however, that the angle ω could be at 0 degrees, such that the tip simply extends from axis J, it could be at 45 degrees, or 180 degrees, where the tip is hooked around such that the tip axis T direction is parallel to axis J. The angle and design of jaw members 108 will be optimized for single jaw tissue retention force during manipulation or tissue apposition. The distance between the pivot aperture 134 and the cam slot 122 dictate the moment arm that translates axial translation to jaw rotation/actuation.

As shown in FIG. 5, each cam slot 122 defines a distal portion 126 and a proximal portion 128 with a middle portion 127 therebetween. The distal portion 126 and the proximal portion 128 of each cam slot 122 is angled relative to the middle portion 127 of each cam slot 122. The proximal portion 128 of each cam slot 122 defines a proximal axis P extending in a first direction. The distal portion 126 of each cam slot 122 defines a distal axis D. The middle portion defines a middle axis M extending in a second direction. The proximal axis P and the distal axis D are both at oblique angle θ relative to the middle axis M. The angle of a respective distal axis D or proximal axis P relative to middle axis M can be fine-tuned to provide optimal tissue clamping force given a user's maximum acceptable input force. While distal axis D and proximal axis P are shown substantially parallel to one another when in the closed configuration, thereby rendering the angle θ between axis P and axis M the same as the angle θ between axis D and axis M, those skilled in the art will readily appreciate that axes P and D may be oblique relative to one another.

With continued reference to FIGS. 5 and 9A, each cam slot 122 includes a distal locking neck 130, e.g., a locking feature, projecting into the cam slot 122 defining a distal locking area 132. The jaw members 108 are in the locked configuration when the second pin 110 is distal relative to the distal locking neck 130 in the distal locking area 132. The distal locking neck 130 includes a protrusion 131 projecting into the cam slot 122. Lock protrusion 131, e.g., a detent, creates a narrowing of cam slot 122 to form the distal locking neck 130 that interferes with the outer diameter of the second pin 110 as it moves axially in the distal direction. Lock protrusion 131 creates an interference fit between pin 110 and distal locking neck 130. The level to which protrusion 131 protrudes into cam slot 122 defines the interference fit and subsequently the force required for pin 110 to overcome said interference and achieve a locked configuration. The mechanism is recoverable, meaning gross plastic deformation of protrusion 131 does not occur, but rather radial flexure of cam slot 122. The initial contact angle between pin 110 and protrusion 131 and the slope following the inflection point (in the distal locking area 132) control the binary behavior of the distal locking neck 130. This slope combined with the level of interference created by protrusion 131 define the locking force.

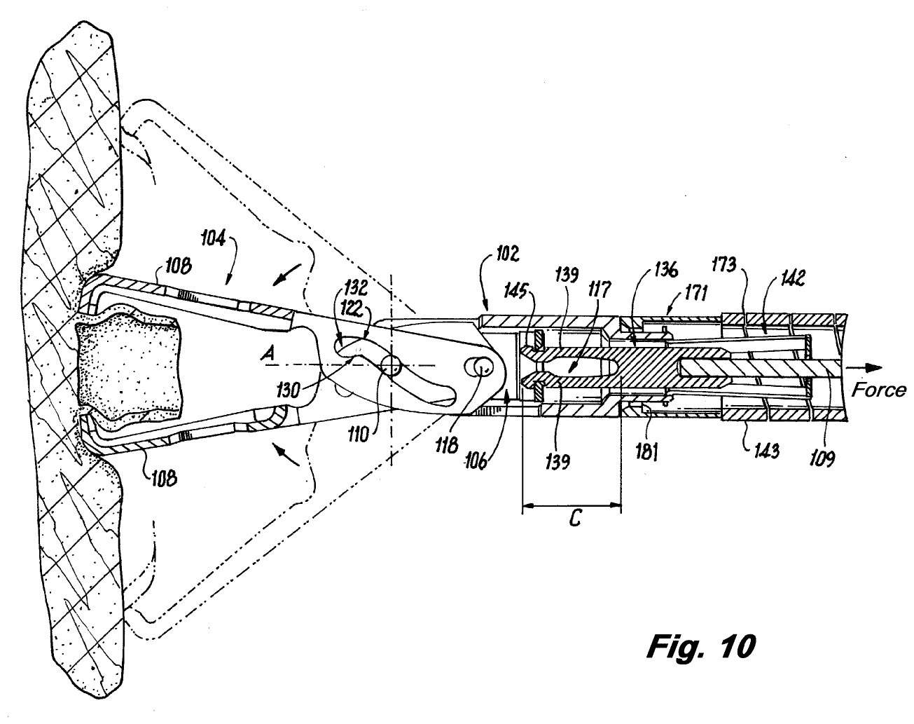

As shown in FIGS. 10-12A, the continued axial translation of pin 110 forces a widening of the cam slot 122 in an elastic manner and creates an additional resistance force on the internal drivetrain, e.g., spring release 136 and shaft spring 142. Once the second pin 110 crests the inflection point on the protrusion 131, it will snap into place distally in front of the protrusion 131, effectively locking the jaws in a closed position. The shape of lock protrusion can vary and can be an arcuate, triangular, or slanted feature. Distal locking neck 130 may also be achieved by reversing the slope of cam slot 122 such that it inflects passed the 0-degree orientation with respect to the axis A of the catheter, described in more detail below. Various embodiments for the distal locking neck are described below in FIGS. 37-39.

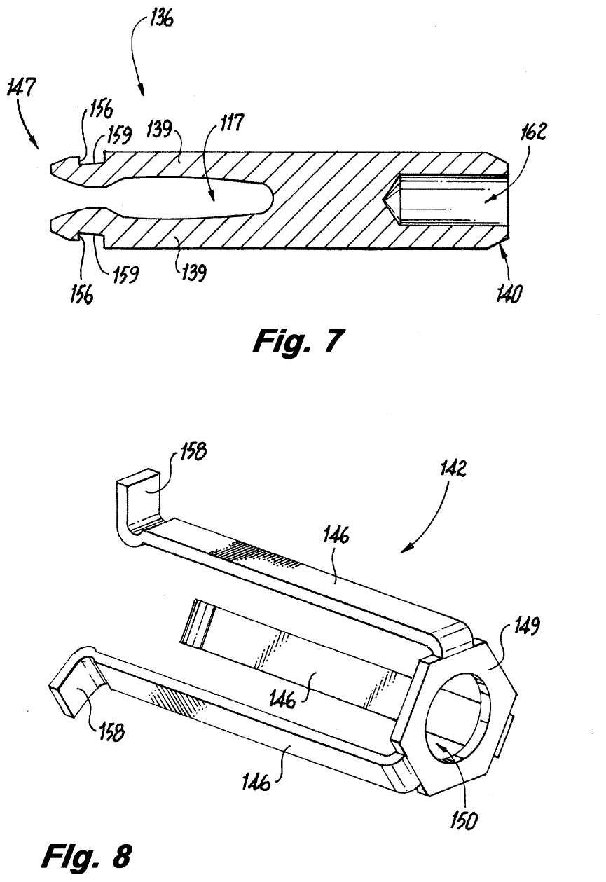

As shown in FIGS. 3 and 6, the jaw adapter yoke 106 includes elongated pin apertures 160 and is operatively connected to the jaw members 108 via first pin 118. Jaw members 108 are configured to rotate about first pin 118. The jaw adapter yoke 106 is circular component with two arms 113 extending towards the distal end of the yoke 106 that form a slot 161 therebetween. The slot 161 allows the proximal portions 116 of the jaw members 108 rotate around first pin 118. One of the apertures 160 is formed in each arm 113 and is in a transverse direction to a longitudinal axis of the jaw adapter yoke 106 and the longitudinal axis A of the catheter body 105. The apertures 160 receive the first pin 118. Pin 118 can be assembled into yoke 160 using press-fitting, orbital riveting, or laser tack welding. The jaw adapter yoke 106 slides linearly inside of distal clip housing 102 to drive jaw members 108 and their respective cam slots 122 axially along the second pin 110. The proximal receiving portion 133 of the jaw adapter yoke 106 includes an inner axially facing surface 135 that mates with a snap feature 141 (described below) at proximally facing mating surface 156 of distal portion 138 of spring release 136, allowing linear force transmission up to a predetermined value. Friction due to an interference fit between an inside surface 129 of yoke 106 and an outer diameter surface 159 of spring release 136 (shown in FIGS. 7 and 11-12A) allows torque transmission from drive wire 109 to the distal subassembly. The proximal receiving portion 133 of the jaw adapter yoke 106 includes an inner boss 137 distal from proximal receiving portion 133 with circumferentially facing flat surfaces 157. Flats 155 of spring release 136 abut flat surfaces 157 to provide a secondary torque transmission between spring release 136 and jaw adapter yoke 106 should the friction from the primary coupling (between outer diameter surface 159 and inside surface 129) be exceeded. A proximal face 125 of yoke 106 contacts the enlarged distally facing surface 164 of spring release 136 allowing axial force in the distal direction to be transmitted from spring release 139 to yoke 106.

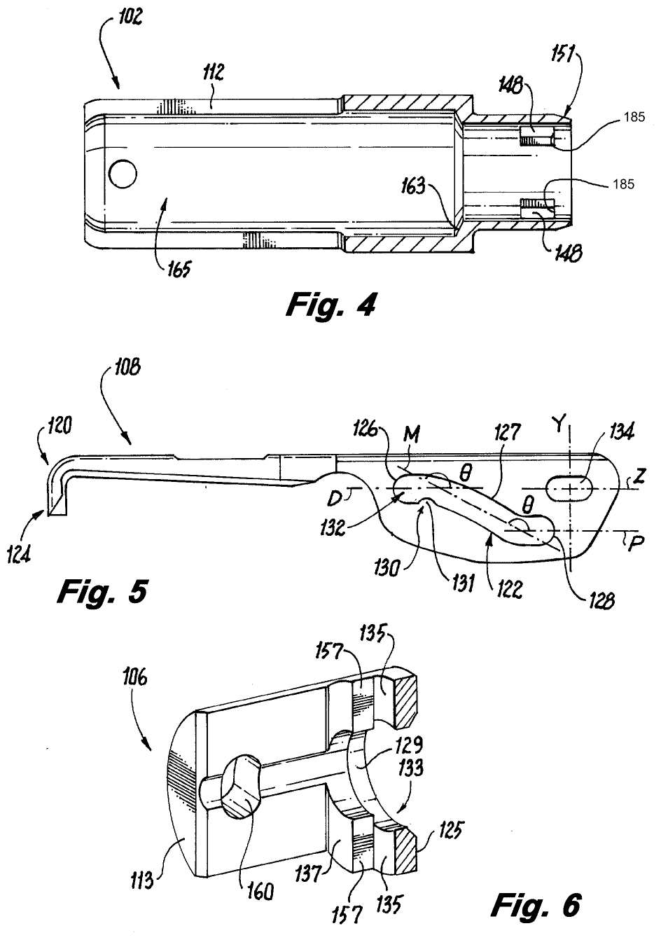

As shown in FIGS. 3-4 and 8-9A, the distal clip housing 102 includes a pair of spaced apart arms 112 defining a slot 114 configured and adapted to provide clearance for respective proximal portions 116 of the jaw members 108 to rotate relative the first pin 118. The distal clip housing 102 connects via a snap fit connection to arms 146 of shaft spring 142 via circumferentially spaced apart apertures 148 defined about a periphery of a proximal end 151 of distal clip housing 102. Distal clip assembly 100 couples to a retention cap 171 via shaft spring 142. Three outward extending flanges 158 from shaft spring 142 intersect the transverse apertures 148 in clip housing 102. Each aperture 148 includes a respective distally facing retaining surface 185 that engages with outwardly extending flanges 158 to resist proximal axial motion of shaft spring 142. While the embodiment of FIGS. 3-4 and 8-9A show the distally facing retaining surface 185 as part of an aperture, it is contemplated that distally facing retaining surface 185 could be part of a circumferential ledge, a detent, or the like. Moreover, while distally facing retaining surface is 185 is shown as being perpendicular to a longitudinal axis of the clip housing 102, it is contemplated that the surface 185 could be oblique to the longitudinal axis of the clip housing. An annular internal ledge 181 in retention cap 171 effectively restrains distal axial motion of shaft spring 142. Retention cap 171 is welded with flat wire coil 173 of distal end 143 of catheter body 105. During assembly of the distal clip assembly 100 to the proximal delivery catheter 101, arms 146 of the shaft spring 142 are pushed radially inward allowing the 90 degree bent tabs (e.g., the outward extending flanges 158 described in more detail below) to seat in respective apertures 148 of distal clip housing 102. An inner surface 165 of distal clip housing 102 further includes a distal facing stop surface 163. The inner diameter surface 165 of distal clip housing 102 allows for axial transmission of jaw adapter yoke 106, until jaw adapter yoke 106 contacts the hard stop created by distal facing stop surface 163.

With reference to FIGS. 3, 7 and 10, the distal portion 138 of the spring release 136 is configured and adapted to transmit axial and rotational force to the jaw adapter yoke 106. The distal portion of the spring release 136 is divided into at least two prongs 139 with a slot 117 therebetween. Prongs 139 are cantilevered arms that terminate in a snap feature 141 at the distal most tip of each prong 139. Snap feature 141 includes a tapered outer diameter surface 145 at the distal tip of each prong 139 and an proximally facing mating surface 156 selectively engageable with an inner surface 135 of the receiving portion 133 of the jaw adapter yoke 106 to prevent axial motion between yoke 106 and spring release 136, up to a certain point. Mating surface 156 allows linear force transmission up to a predetermined value. Different deployment forces can be easily achieved by varying dimensions of spring release 136. Varying the diameter of mating surface 156 or other dimensions of snap feature 141 will have great effect on the release force, as will changing the length C of prongs 139 (which thereby adjusts the length of the slot 117 between prongs 139). The slot 117 must be long enough so as to not cause plastic deformation during the assembly process. Slot 117 may also be non-axisymmetric so as to stiffen one prong 139 relative to the other. This could bias the release mechanism so that one prong 139 always deflects first, increasing repeatability in force design. Those skilled in the art will also readily appreciate that receiving portion 133 could also have an inner surface with a square cross-section such that flat outer surfaces 155 on prongs 139 interface with inner diameter surface to transmit torque.

With continued reference to FIGS. 3 and 9A-9C, the drive wire 109 is mechanically coupled to a proximal portion 140 of the spring release 136 to transmit linear and rotational motion from the drive wire 109 to the jaw adapter yoke 106. The proximally facing end of spring release 136 has a receiving bore 162 opening in the proximal direction for receiving and coupling the drive wire 109 to the spring release 136. Drive wire 109 can be coupled via a laser weld, crimp, swage, or interference fit. The proximal portion 140 of the spring release 136 being the portion proximal relative to prongs 139. The rotation of drive wire 109 about the longitudinal axis A drives rotation of spring release 136, jaw adapter yoke 106, distal clip housing 102 and jaw assembly 104 about the longitudinal axis A relative to catheter body 105.

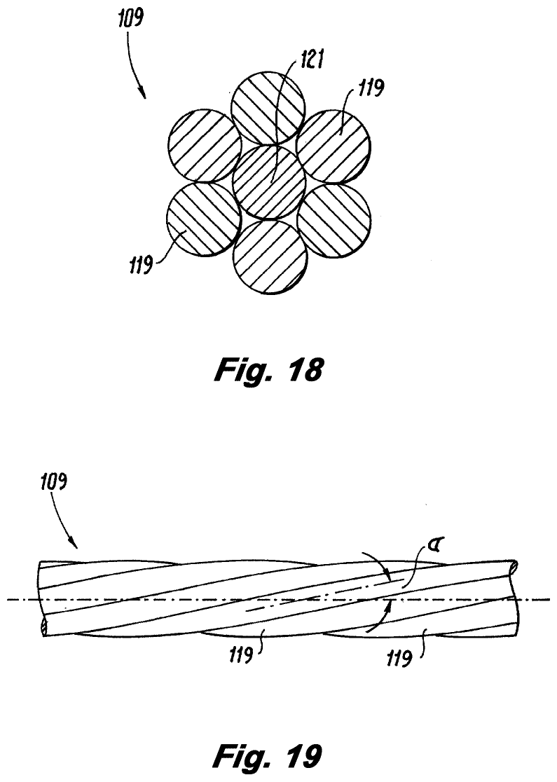

As shown in FIGS. 18-19, drive wire 109 is comprised of one central filar 121 and six outer filars 119. The flexibility and torsional capability of the overall drive wire 109 is a function of geometric construction of the filars 119, 121, a pitch angle α of the outer wind, the hardness and alloy of the wire, processing techniques during wire forming and winding, and lay pattern. In the embodiment of FIGS. 1-3 and 18-19, the selected configuration for drive wire 109 is a 1×7 wire rope, although 1×3, 1×12, 1×19, and 7×7 are all alternative options if the outer diameter remains consistent. The 1×7 construction for drive wire 109 allows for maximal flexibility while still maintaining a high cross-sectional density and good torsional properties with respect to overall wire diameter. Of note, the central filar 121 is between 10% and 30% larger than the outer filars 119 to facilitate higher strength and control the pitch. While all filars 119, 121, will not have the same material properties due to the forming and drawing process, certain initial filar properties are required such as surface roughness below 16 rma, preferably below 6 rma. The pitch angle α is between 9° and °30 and can be tailored depending on the flexibility needed.

As shown in FIGS. 3, 8 and 10, the arms 146 of the shaft spring 142 can be laser cut, creating at least one cantilever arm 146. Outwardly extending flanges 158 are 90-degree tabs bent outward that mate with respective circumferentially spaced apart apertures 148 of housing 102. Arms 146 are parallel or substantially parallel to one another and are in cantilever arrangement with the annular portion 109 and through-hole 150. Outwardly extending flanges 158 extend radially or substantially radially outward from respective arms. Arms 146 are elastic enough to deflect inwards until the bottoms of outwardly extending flanges 158 are tangent to longitudinal axis without permanent deformation. Outwardly extending flanges 158 must be plastically deformed to disengage from the assembled configuration. The spring release 136 is positioned at least partially within the shaft spring 142. Annular portion 149 of the shaft spring 142 is positioned around the drive wire 109.

With reference now to FIGS. 9A-9C, some of the various configurations of device 10 are shown. In FIGS. 9A-9C, the device 10 is in an open configuration and the jaw members 108 and their respective cam slots 122 are translated in a distal most position relative to second pin 110. Jaw members 108 open by applying compression to the inner drivetrain (yoke 106, first pin 118, spring release 136 and control wire 109) and tension to the outer drivetrain (clip housing 102, shaft spring 142 retention cap 171 and catheter 105), creating relative motion between the distal clip housing 102 yoke 106 and jaw members 108, and second pin 110 to move along cam slots 122. Jaws 108 close by applying tension to the inner drivetrain and compression to the outer drivetrain, creating relative motion between the distal clip housing 102 and second pin 110, and yoke 106 and jaw members 108 to move along cam slots 122 as jaws move in the proximal direction.

In FIG. 9B, the device 10 is in an open configuration with the jaw members 108 canted in a proximal direction. In this canted configuration, first pin 118 is able to move in a downward direction perpendicular to the longitudinal axis A (e.g. downwards as oriented in FIG. 9B) due to elongated pin apertures 160. In this way, the distal tip 124 of the bottom jaw member 108 is in a more distal position P₂ along longitudinal axis A as compared to a position P₁ of distal tip 124 of the top jaw member 108, and a first angle β between top jaw member 108 and the longitudinal axis A is slightly larger than a second angle γ between bottom jaw member 108 and the longitudinal axis A, creating the proximally canted orientation. In FIG. 9C, the device 10 is in an open configuration with the jaw members 108 canted in a distal direction. In this canted configuration, first pin 118 is able to move in an upwards direction perpendicular to the longitudinal axis A (e.g. upwards as oriented in FIG. 9B) due to elongated pin apertures 160. In this way, position P₁ of distal tip 124 of the top jaw member 108 is in a more distal position along to longitudinal axis A as compared to position P₂ of distal tip 124 of the bottom jaw member 108, and first angle β between top jaw member 108 and the longitudinal axis A is slightly smaller than second angle γ between bottom jaw member 108 and the longitudinal axis A, creating the distally canted orientation. Those skilled in the art will readily appreciate that the cant angle of the jaw members can vary over a continuous range of angles between the position of FIG. 9B and FIG. 9C. In other words, there are more than just two canted positions. In FIG. 9A, where the jaws are in an un-canted position, first angle β and second angle γ are congruent with one another.

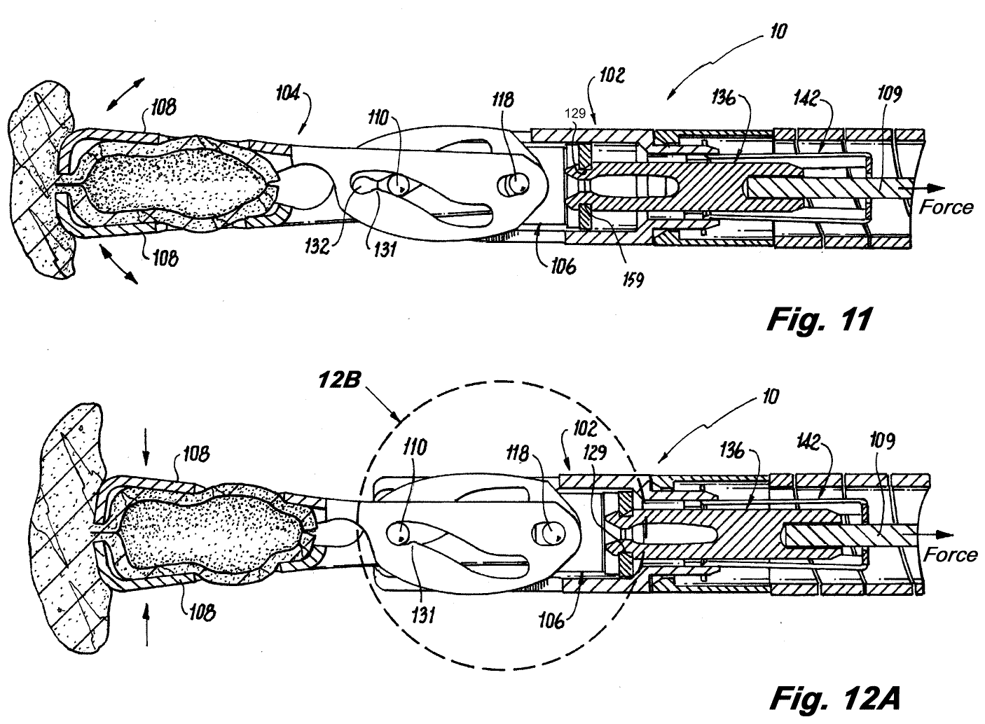

In FIG. 10, the device 10 is shown between the open and the closed configuration and the first pin 118, yoke 106 and spring release 136 are translated in a more proximal position relative to distal clip housing 102 and relative to the open configuration of FIGS. 9A-9C. Additionally, in FIG. 10 as compared to FIGS. 9A-9C, cam slots 122 slid proximally along with their jaw members 108 relative to second pin 110, such that second pin 110 is in a more distal portion of the cam slots 122. In FIG. 10, second pin 110 is still proximal of the locking neck 130 and the protrusion 131. In FIG. 11, the closed configuration is shown. In the closed configuration, the respective distal tips 124 of the jaw members 108 are approximated towards one another to grasp tissue 15 (but not necessarily in abutment with one another). In FIG. 11, the device 10 is closed, but not locked, meaning that second pin 110 is still proximal of the protrusion 131 and that jaw members can still be rotated back open, if needed.

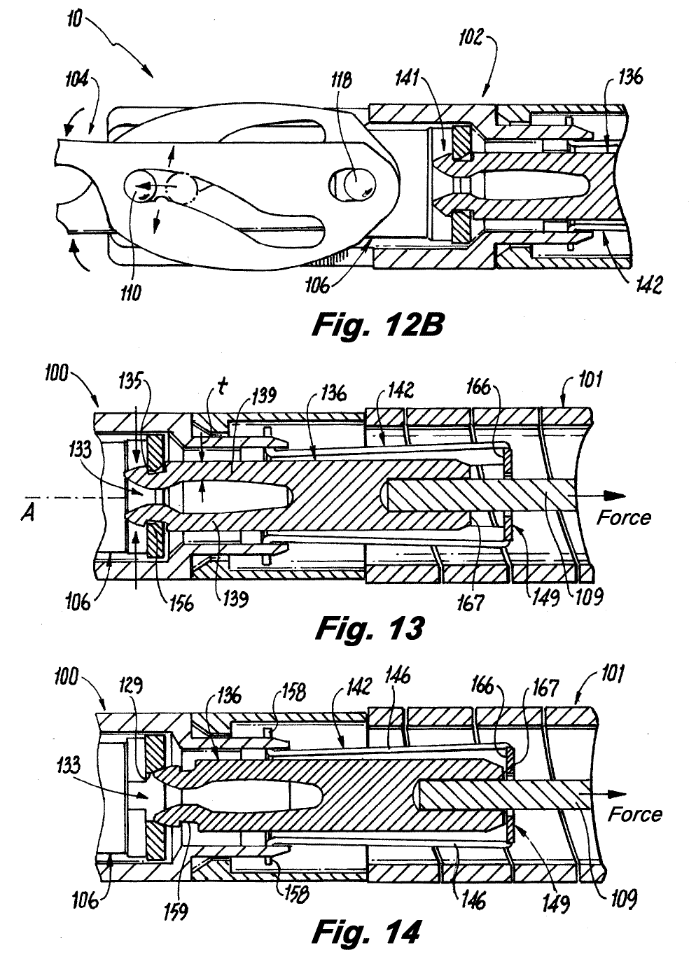

With reference now to FIGS. 11-12B, the device 10 transitions from a closed configuration (FIG. 11) to a locked configuration (FIGS. 12A-12B) as control wire 109 is pulled proximally. From the closed configuration, the continued axial translation of second pin 110 forces a widening of cam slot 122 in an elastic manner (as indicated schematically by the arrows in FIG. 12B) and creates a additional resistance force on the internal drivetrain. Once second pin 110 crests the inflection point on the protrusion 131, it will snap into place behind the protrusion 131, effectively locking the jaw members 108 in a closed position, shown in FIGS. 12A-12B. Because the drive wire 109 only transmits limited compression, a user will generally not be able to translate sufficient force from the handle distally to second pin 110 relative to the protrusion 131 to unlock the second pin 110 from the locking area 132. In the locking configuration, the second pin 110 is in a distal position relative to the locking necks 130 and their respective protrusions 131. In the locked configuration, the second pin 110 is within the distal locking area 132 of each cam slot 122.

As shown in FIGS. 12B-17, once the second pin 110 is in the distal locking area 132, further axial movement of the spring release 136 in a proximal direction (e.g., away from the tissue 15) acts to “fire” the distal clip assembly 100 by releasing the distal clip assembly 100 from the proximal delivery catheter 101. The further linear motion of the spring release 136 in the proximal direction puts the spring release 136 in tension against jaw adapter yoke 106 due to abutment between mating surface 156 of the snap feature 141 and the inner surface 135 of the receiving portion 133. This tension causes each prong 139 to act as a spring and deflect inwardly, shown schematically by the inwardly pointing arrows in FIG. 13, and release from the receiving portion 133. The release force required to detach spring release 136 from the adapter yoke 106 can be tuned by adjusting the length C (shown in FIG. 10) of each prong 139, the thickness t of each prong 139, and/or the angle of mating surface 156 relative to the longitudinal axis A, shown in FIG. 18. The angle of mating surface 156 relative to longitudinal axis A is shown as 90 degrees, but can range from 30 to 90 degrees, e.g., 45 degrees. The ratio of length C to thickness t can range from 8:1 to 10:1, e.g., 9:1. These dimensions provide the desired elastic behavior to ensure consistent release.

As shown in FIGS. 14-17, as the spring release 136 moves proximally relative to the jaw adapter yoke 106, it also moves proximally relative to the shaft spring 142, thereby causing abutment between a proximally facing surface 167 of spring release 136 and a distally facing surface 166 of annular portion 149 of the shaft spring 142. The abutting causes shaft spring 142 to translate along the longitudinal axis A in a proximal direction, eventually bending each outwardly extending flange 158 inwardly toward longitudinal axis A to disengage each outwardly extending flange 158 from their respective circumferentially spaced apart aperture 148 of the distal clip housing 102. The maximum diameter of proximally facing surface 167 of spring release 136 is greater than the inner diameter of through-hole 150. Full disengagement (e.g., “firing”) of the distal clip assembly 100, shown in FIG. 16, is realized through both the inward deflection and release of the prongs 139 of spring release 136 and the inward bending and release of the flanges 158 of shaft spring 142.

With continued reference to FIGS. 14-17, a single spring component, spring release 136, disengages two springs (prongs 139 and arms 146) generating an improved disconnect mechanism that enhances the ability to reposition the clip assembly 100 prior to deployment by simplifying the feedback to the user into a single tactile signal, decreasing the likelihood of user confusion as to whether or not the clip 100 has fully deployed. In the embodiment of FIGS. 14-17, prongs 139 begin deflecting first, but the bending of the outwardly extending flanges 158 occurs simultaneously with the release of prongs 139 of spring release 136.

A method for firing a hemostatic clip assembly, e.g., distal clip assembly 100, includes positioning the distal clip assembly proximate to a target location, e.g., near tissue 15 as shown in FIG. 14, and translating an actuation portion, e.g., actuation portion 115, of a proximal handle assembly, e.g., proximal handle assembly 103, of a proximal delivery catheter, e.g., proximal delivery catheter 101, relative to a grasping portion, e.g., grasping portion 107, of the proximal handle assembly in at least one of a proximal direction or a distal direction. The actuation portion is operatively connected to a jaw adapter yoke, e.g., jaw adapter yoke 106, via a drive wire, e.g., drive wire 109, and a spring release, e.g., spring release 136 to transmit linear motion to the jaw adapter yoke. The linear motion of the jaw adapter yoke transmits a linear component of motion to at least one jaw member, e.g., jaw member 108, and its cam slot, e.g., cam slot 122, to translate the cam slot along a second pin, e.g., second pin 110, connecting between at least one of the jaw members and the distal clip housing, thereby rotating at least one of the jaw members about the first pin between an open configuration and a closed configuration, and/or between the closed configuration and the open configuration.

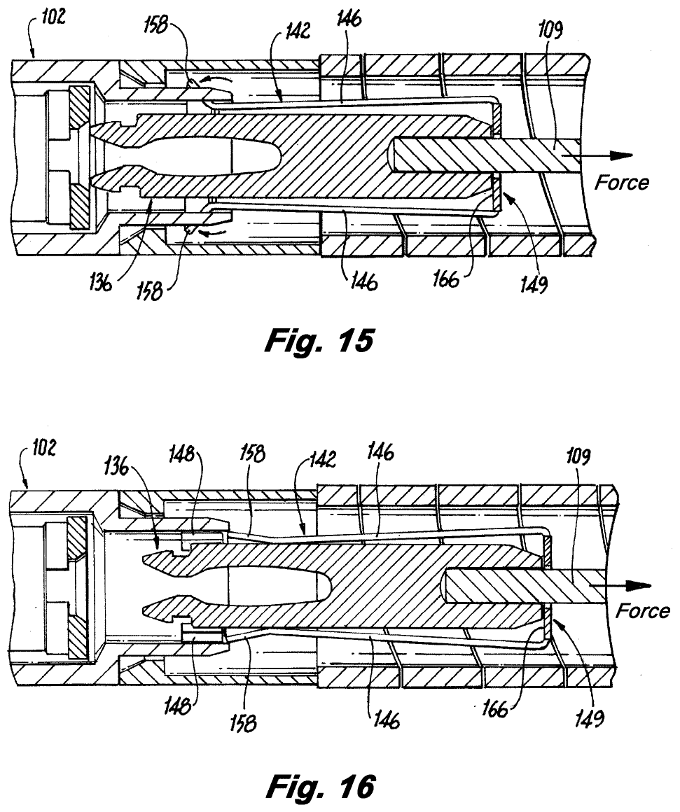

The method includes translating the actuation portion in the proximal direction to transmit the linear motion in the proximal direction to the cam slot, as shown in FIG. 15, to lock the second pin behind a lock protrusion, e.g., lock protrusion 131, of the cam slot to lock at least one of the jaw members in a locked configuration, as shown in FIG. 12A. Translating the actuation portion includes translating the actuation portion in the proximal direction to transmit linear motion in the proximal direction to the spring release, as shown in FIG. 13. The linear motion in a proximal direction de-coupling a distal portion, e.g., distal portion 138, of the spring release from a receiving portion, e.g., receiving portion 133, of the jaw adapter yoke, as shown in FIGS. 13-14. Translating the actuation portion includes translating the spring release in the proximal direction causing abutting between the spring release and an annular portion, e.g. annular portion 149, of a shaft spring, e.g. shaft spring 142. The abutting causes an outwardly extending flange, e.g. outwardly extending flange 158, to bend inward and disengage from a proximal end, e.g. proximal end 151, of a distal clip housing, e.g. distal clip housing 102.

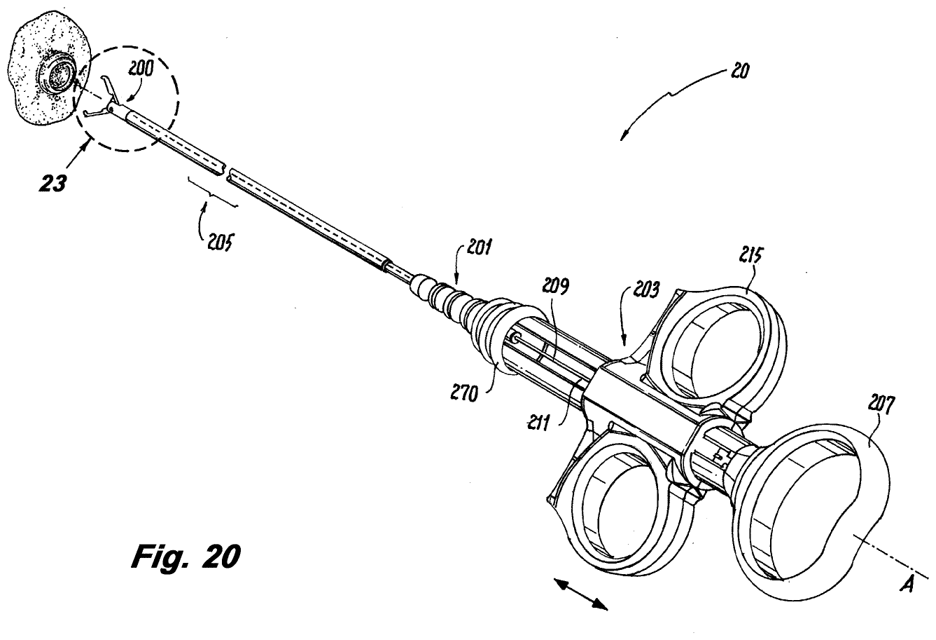

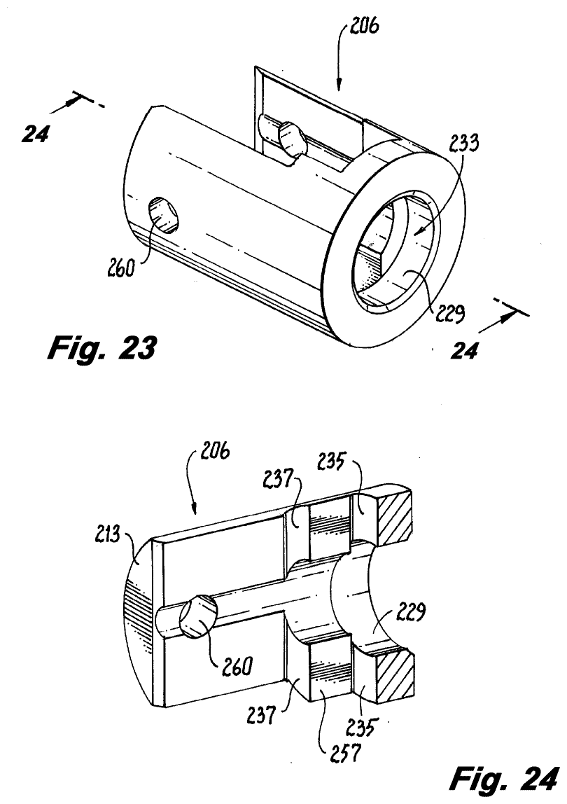

As shown in FIGS. 20-25, another embodiment of a surgical device 20 for applying a hemostatic clip assembly 200 includes proximal delivery catheter 201 and the distal clip assembly 200. Delivery catheter 201 of FIG. 20 is the same as delivery catheter 101 shown in FIG. 1 and described above. As such, the description provided above for delivery catheter 101, its proximal handle assembly 103, elongated catheter body 105, drive wire 109, actuation portion 115, grasping portion 107, and the like readily apply to delivery catheter 201 of FIG. 20. The distal clip assembly 200, e.g., a hemostasis clip, separates from the delivery catheter 201 to function as a short-term implant to stop and prevent re-bleeding, or in procedures such as ampullectomy, polypectomy, tissue repair and correction of other tissue defects. Such procedures are the same as those described above in the context of FIG. 1.

With continued reference to FIGS. 20-25, the proximal delivery catheter 201 has a proximal handle assembly 203 and an elongated catheter body 205 extending distally from the proximal handle assembly 203. The elongated catheter body 205 defines a longitudinal axis A. The proximal handle assembly 203 includes an actuation portion 215 coupled to a proximal end 211 of the drive wire 209, and a grasping portion 207. Grasping portion 207 and actuation portion 215 are configured and adapted to rotate relative to a cap 270 and catheter body 205, thereby also rotating drive wire 209. Internal annular slots on the distal portion of grasping portion 207 interact with annular tabs on inside diameter of end cap 270 to prevent axial motion of actuation portion 215 and grasping portion but allow rotation.

As shown in FIGS. 20-25 and 45, the proximal delivery catheter 201 includes a shaft spring 242 between a proximal end of the distal clip assembly 200 and a distal end 243 of the catheter body 205. Shaft spring 242 includes arms 246, outward extending flanges 258, an annular portion 249. Shaft spring 242 is similar to shaft spring 142 except that the length of shaft spring 242 is shorter than that of shaft spring 142. The distal clip assembly 200 includes a distal clip housing 202 and a jaw assembly 204 slidably connected to the distal clip housing 202. Distal clip housing 202 and jaw assembly 204 are similar to distal clip housing 102 and jaw assembly 104, described above. The description provided above for jaw assembly 104 and distal clip housing 102 readily apply to distal clip housing 202 and jaw assembly 204. The distal clip assembly 200 includes a jaw adapter yoke 206 positioned within the distal clip housing 202 and slidable relative to the distal clip housing 202. The jaw assembly 204 has a pair of cooperating jaw members 208 rotatably connected to the jaw adapter yoke 206 at a first pin 218 and slidably connected to the distal clip housing 202 by a second pin 210, similar to first pin 118 and second pin 110 of device 10 described above. The proximal delivery catheter 201 includes a spring release 236 having a distal portion similar to distal portion 138 configured and adapted to be received within a proximal receiving portion 233 of the jaw adapter yoke 206.

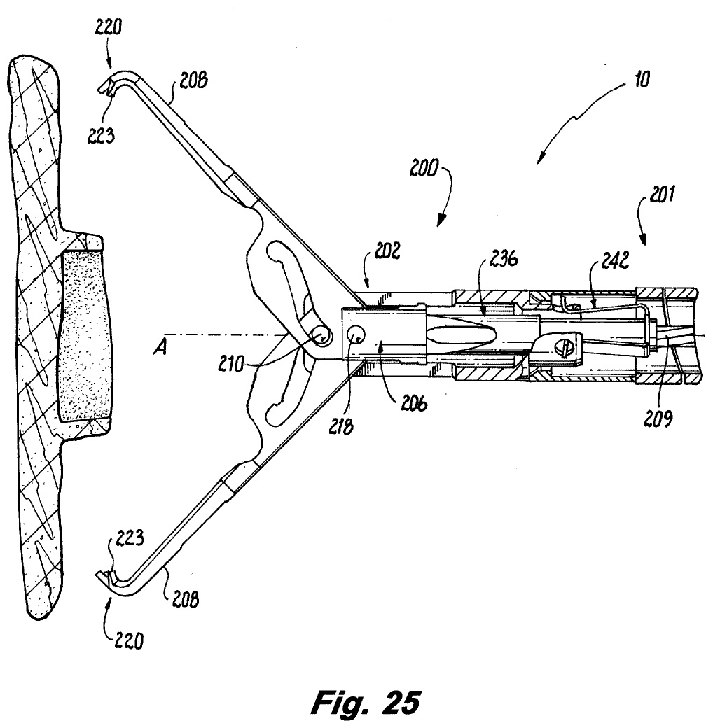

As shown in FIGS. 25-28, each jaw member 208 includes a proximal body portion 216 and a distal end effector 220. Each jaw member 208 is essentially the same as jaw members 108 except for the specific teeth 223 used on distal end effector 220. A respective cam slot 222 is the same as cam slot 122. The drive functionality of jaw members 208 (e.g. how they open and close) is the same as jaw members 108. Each jaw member 208 includes a pivot aperture 234, similar to pivot aperture 134, configured and adapted to receive the first pin 218. The dimensions, angles, shapes, and variability described above for jaw members 108 and pivot apertures 134 readily apply to jaw members 208 and pivot apertures 234. The distal end effectors 220 of each jaw member 208 include teeth 223 that interlock with teeth of an abutting opposite jaw member. Teeth 223 are slightly different than teeth 123, in that teeth 123 include an edge tooth pointing in a different direction from the other teeth. Teeth 223 positioned proximate to the side edge of their respective jaw member 208 optimize and maximize single-jaw tissue retention force during manipulation or tissue apposition, so as to catch tissue/defects easily, especially in non-perpendicular approaches to tissue. The end effector 220 of each jaw member 208 includes an internal concave surface 253 and outer convex surface 286, similar to inner surface 153 and outer surface 186.

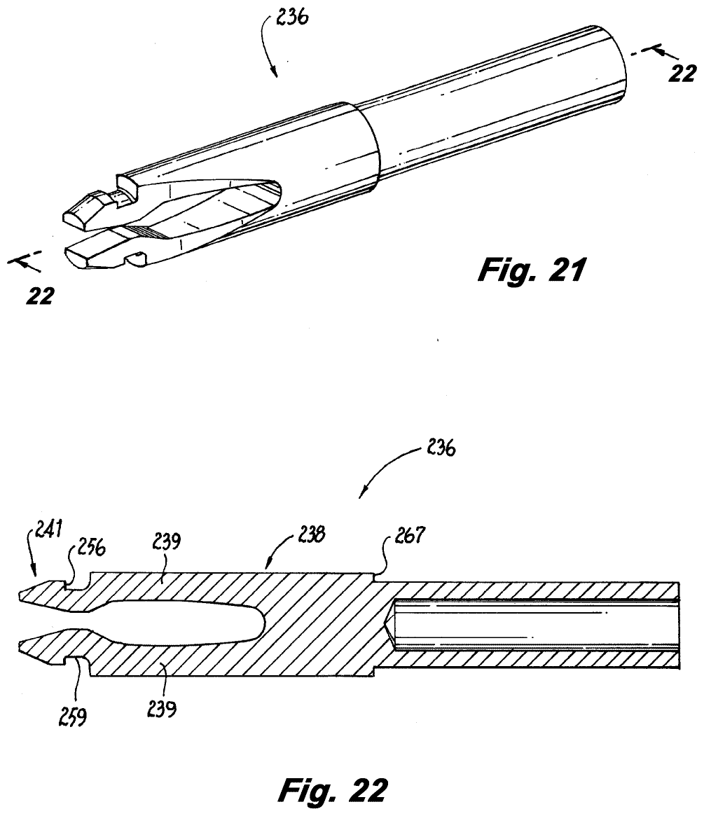

With continued reference to FIGS. 25-28, each cam slot 222 is similar to cam slot 122. Each cam slot 222 includes a distal locking neck 230, e.g., a locking feature, projecting into the cam slot 222 defining a distal locking area 232. The locking sequence, functionality and position of cam slot 222 relative to pin 210 is the same as cam slot 122. The distal locking neck 230 includes a protrusion 231 projecting into the cam slot 222, the same as protrusion 131. As shown in FIGS. 21-24 and 29-30, the jaw adapter yoke 206 is similar to jaw adapter yoke 106 except that adapter yoke 106 includes pin apertures 260 that have equivalent dimensions in the axial and transverse to axial directions. This deviates from pin apertures 160, which are elongated in the axial direction. The proximal receiving portion 233 is the same as proximal receiving portion 133. Receiving portion 233 includes an inner axially facing surface 235 that mates with a snap feature 241 (described below) at proximally facing mating surface 256 of distal portion 238 of spring release 236, allowing linear force transmission up to a predetermined value. Friction due to an interference fit between an inside surface 229 of yoke 206 and an outer diameter surface 259 of spring release 236 allows torque transmission from drive wire 209 to the distal subassembly. The proximal receiving portion 233 includes a boss 237, similar to boss 137, with circumferentially facing flat surfaces 257. Flats 255, similar to flats 155 of spring release 136, abut flat surfaces 257 to provide a secondary torque transmission between spring release 236 and jaw adapter yoke 206, similar to device 10. A proximal face 225 of yoke 206 contacts the enlarged distally facing surface 264 of spring release 236 allowing axial force in the distal direction to be transmitted from spring release 239 to yoke 206.

As shown in FIGS. 21-22 and 29-30, the distal clip housing 202 is essentially the same as distal clip housing 102. The distal clip housing 202 connects via a snap fit connection to arms 246 of shaft spring 242 via circumferentially spaced apart apertures 248. Distal clip assembly 200 couples to a retention cap 217 via shaft spring 242, similar to clip assembly 100 and retention cap 171. Distal clip housing 202 further includes a distal facing stop surface 263, similar to surface 163. Spring release 236 is similar to spring release 136, except that spring release 236 includes a boss having a proximally facing surface 267 that abuts a distally facing surface 266 of shaft spring 242, instead of a proximal-most end surface 167 of spring release 136. is configured and adapted to transmit axial and rotational force to the jaw adapter yoke 106. The distal portion of the spring release 236 is divided into at least two prongs 239, similar to spring release 136. Prongs 239 are cantilevered arms that terminate in a snap feature 241 at the distal most tip of each prong 239. Snap feature 241 is similar to 141. Varying the diameter of mating surface 256 or other dimensions of snap feature 241 will have great effect on the release force, as will changing the length C of prongs 239 (which thereby adjusts the length of the slot between prongs 239), similar to the slot 117 and prongs 139 of spring release 136.

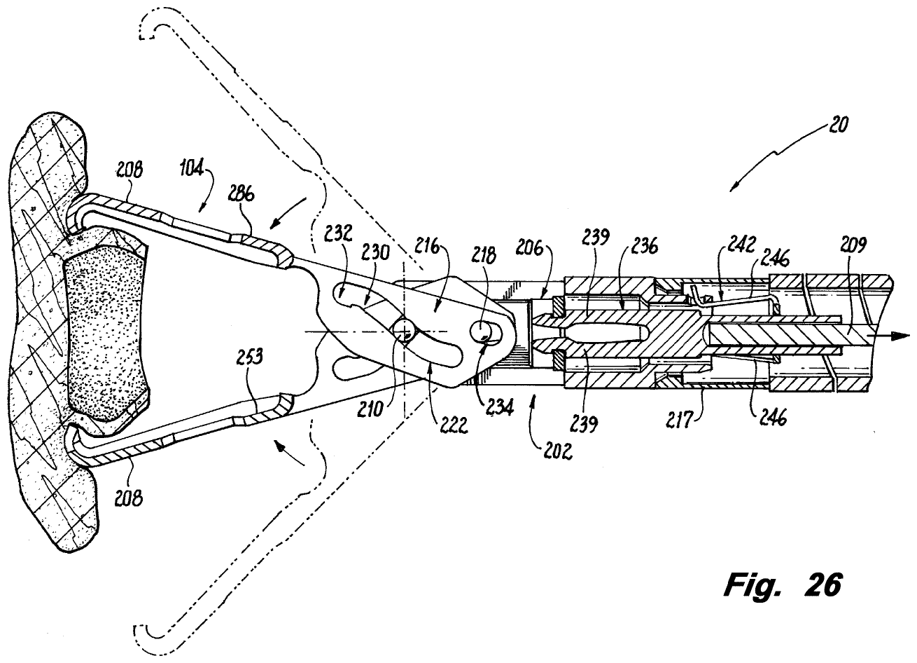

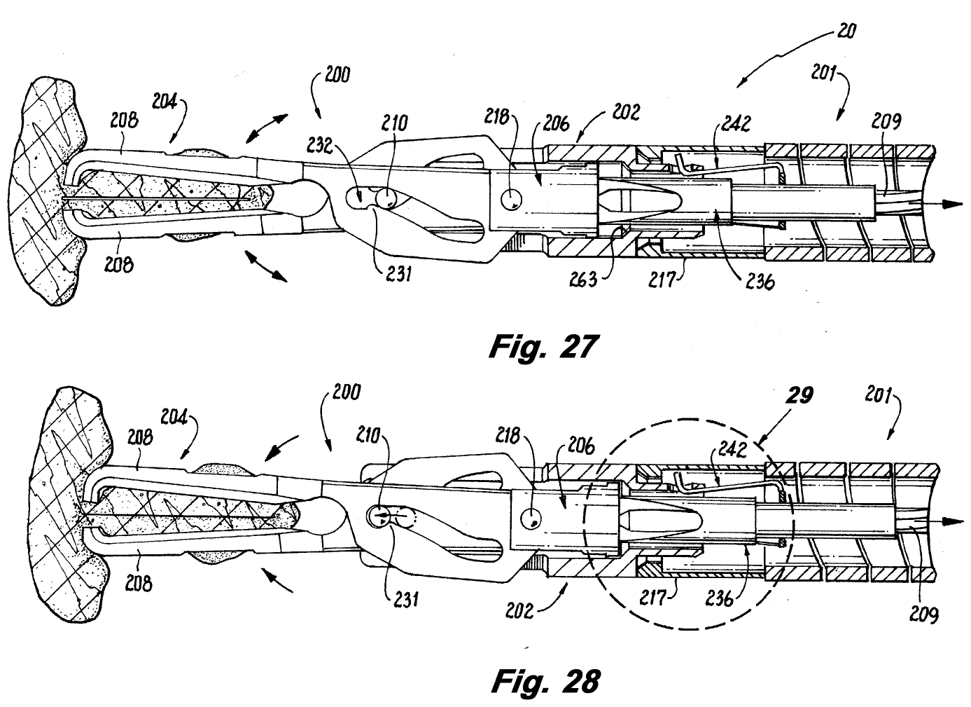

With continued reference to FIGS. 26-30, the drive wire 209 is mechanically coupled to a proximal portion 240 of the spring release 236, similar to spring release 136 and drive wire 109. In FIG. 26, the device 20 is in a partially open configuration and the jaw members 208 and their respective cam slots 222 are translated in a mid-way position relative to second pin 210. Jaws 208 open and close in a similar manner to that described above for jaws 108, except that jaw members 208 do not have the canting functionality like jaw members 108. In FIG. 27, the closed configuration is shown. In the closed configuration, the respective distal tips 224 of the jaw members 208 are approximated towards one another to grasp tissue 15 (but not necessarily in abutment with one another). In FIG. 28, the device 20 transitions from a closed configuration to a locked configuration as control wire 209 is pulled proximally, similar to device 10.

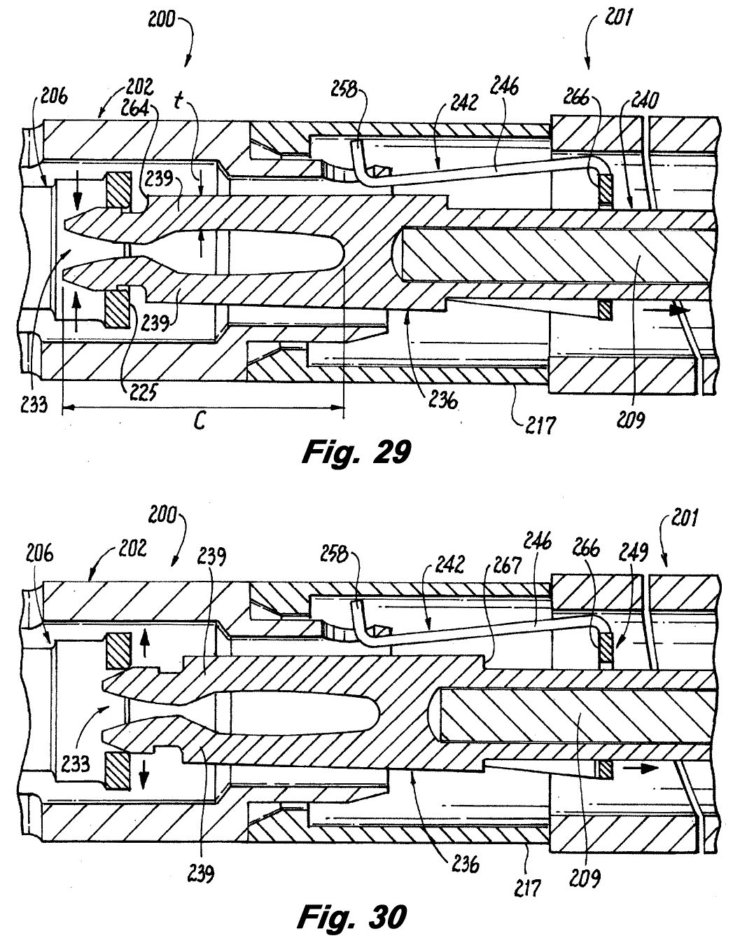

As shown in FIGS. 28-31, once the second pin 210 is in the distal locking area 232, further axial movement of the spring release 236 in a proximal direction (e.g., away from the tissue 15) acts to “fire” the distal clip assembly 200 by releasing the distal clip assembly 200 from the proximal delivery catheter 201, similar to the firing described above for device 10. The release force required to detach spring release 236 from the adapter yoke 206 can be tuned by adjusting the length C (shown in FIG. 29) of each prong 239, the thickness t of each prong 239, and/or the angle of mating surface 256 relative to the longitudinal axis A, similar to spring release 136 described above.

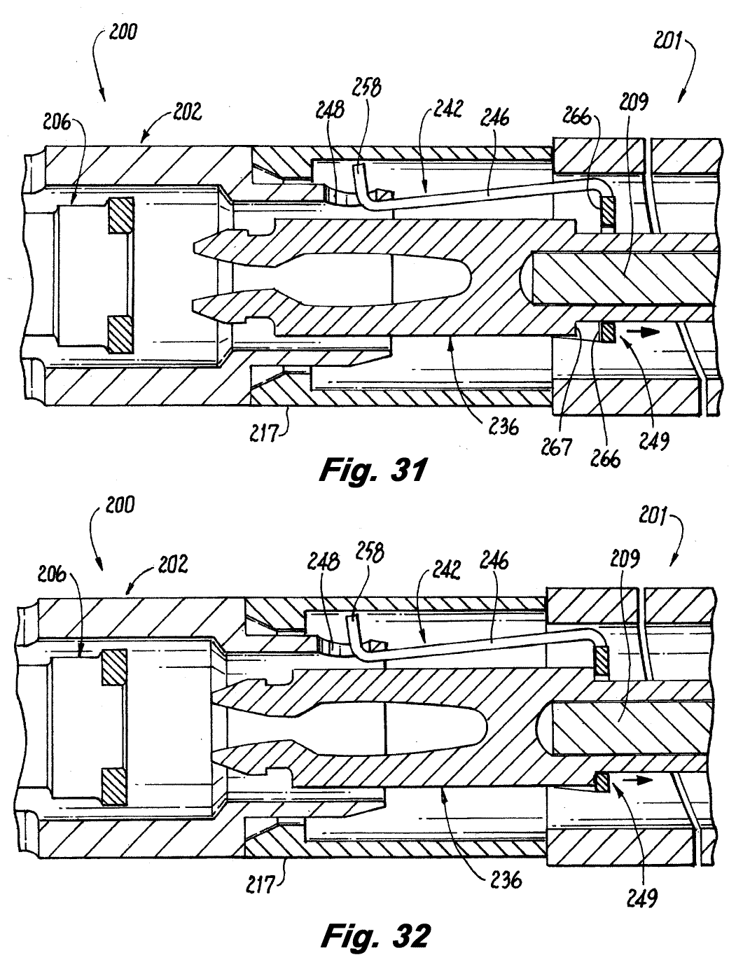

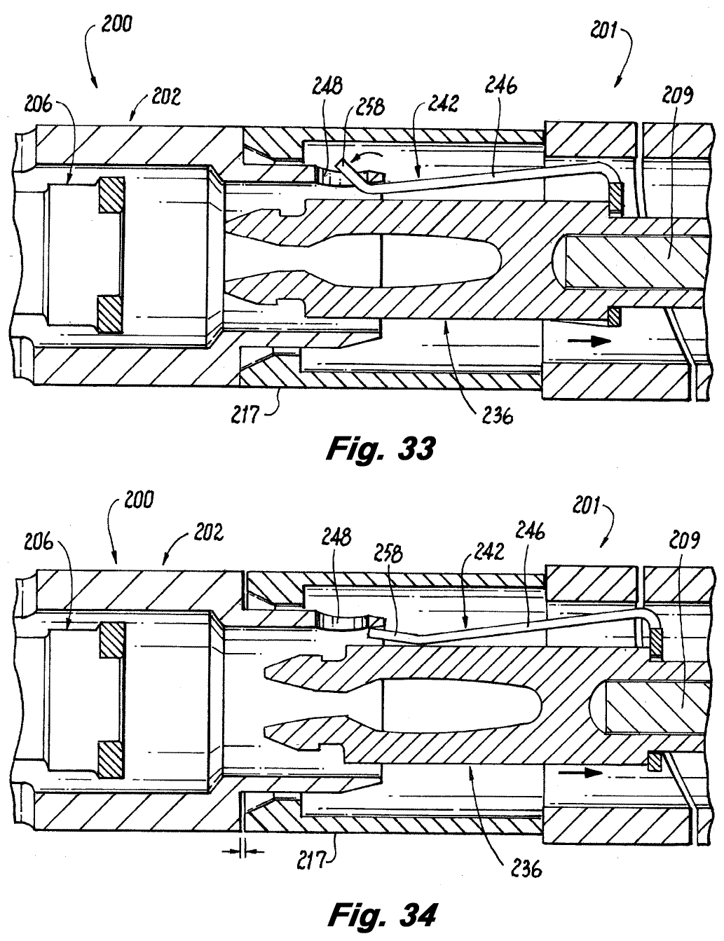

As shown in FIGS. 31-34, as the spring release 236 moves proximally relative to the jaw adapter yoke 206, it also moves proximally relative to the shaft spring 242, thereby causing abutment between a proximally facing surface 267 of spring release 236 and a distally facing surface 266 of annular portion 249 of the shaft spring 242, similar to spring release 136 and shaft spring 242. A single spring component, spring release 236, disengages two springs (prongs 139 and arms 146) generating an improved disconnect mechanism that enhances the ability to reposition the clip assembly 200 prior to deployment by simplifying the feedback to the user into a single tactile signal, decreasing the likelihood of user confusion as to whether or not the clip 100 has fully deployed, similar to device 10.

Clip assemblies 100 and 200 possess several improvements over other designs in the prior art. The distal clip assemblies 100 and 200 offer a simplified design where the external release and rotation joint between the clip assembly and delivery catheter are one and the same, allowing for fewer components to achieve desired performance. This enables a method of assembly that is much simpler than traditional designs. The clip assemblies 100 and 200 can be built independent of the proximal delivery catheter, enabling full automation when manufacturing the distal end. In addition, both the external and internal drive trains connect via a simple snap connection, eliminating potential fragmentation due to frangible elements, this simplifies clip assemblies 100 and 200 in that the clips do not require special machinery or highly skilled labor to assemble. The snap fit engagement between housing 102 or 202 and arms 146 or 246 greatly expedites the assembly process, and offers less room for error than alternate forms of joining (i.e. welding, deformation based).

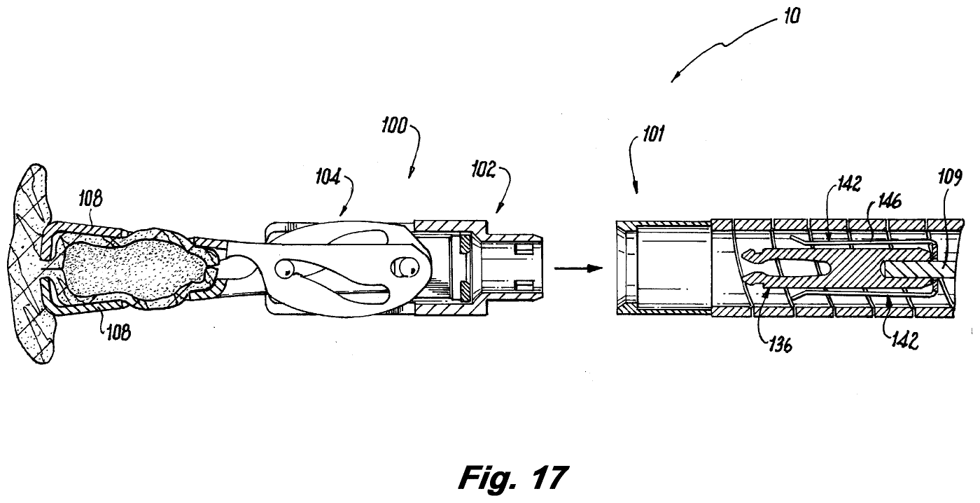

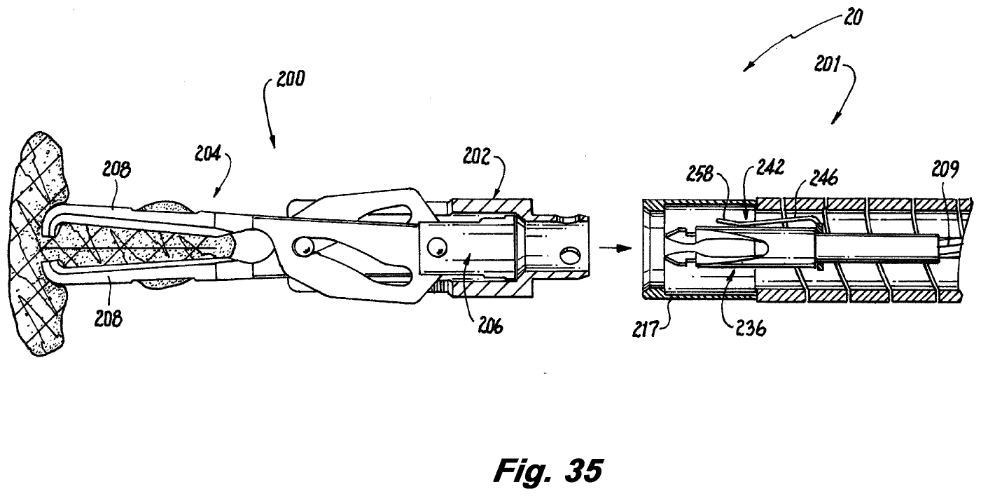

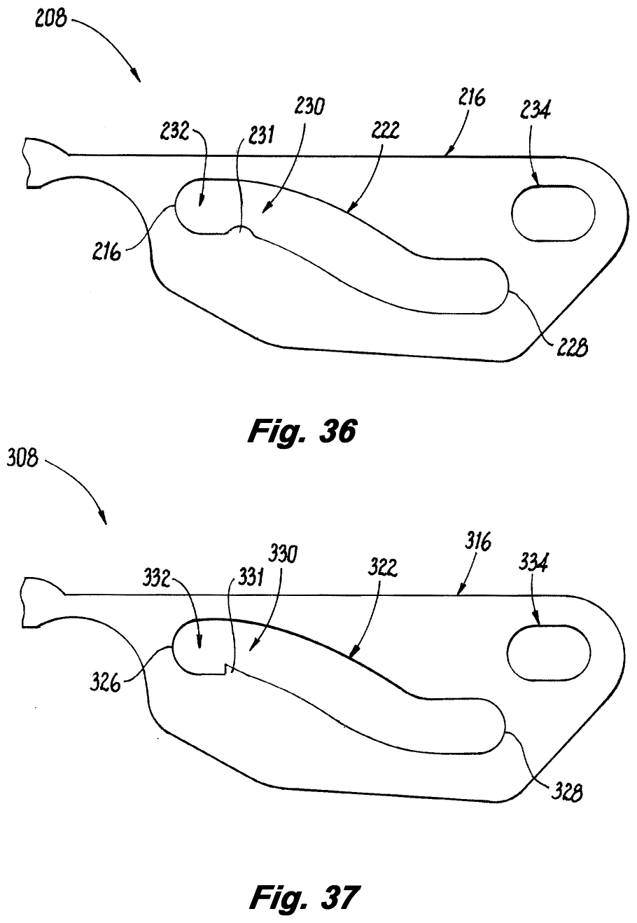

Because there are fewer components, less space is needed in the distal assembly, allowing for a shorter clip body. The shorter clip “stem” or overall length of deployed clip relative to jaw size is seen as an improvement. In addition to the simplified user feedback, it also makes accidental deployment of the clip assemblies 100 and 200 less likely, as fewer components are used to realize disengagement. Because there are fewer components, less space is needed in the assemblies 100 and 200, allowing for a shorter clip body. The shorter clip “stem” or overall length of deployed clip relative to jaw size is seen as an improvement. As shown in FIGS. 17 and 35, after firing, proximal delivery catheters 101 and 201 can then be removed from the surgical site, leaving the distal clip assemblies 100 and 200 to function as short-term implants.

Furthermore, after firing, spring release 136 or 236 can continue to move proximally and recede into catheter body 105 or 205. For the embodiment of FIG. 1, the near-concurrent prong 139 release and bending of outwardly extending flanges 158 firing is a sudden reduction in force required on actuator 115. A small gap occurs between the decoupling of spring release 136 or 236 and decoupling of shaft spring 105 or 205 to avoid both forces stacking additively in relation to the users perception. The gap is minute as to be imperceptible to the end user and the force of 105 or 205 decoupling being significantly lower than the force of 136 or 236 decoupling leads to automatic decoupling of the secondary joint (the decoupling of 105 or 205) if sufficient force has been applied to the first joint to realize decoupling. Contrary to some designs seen in the prior art, where there may be a stop flange on a spring release to prevent spring release from receding further, present embodiments provide the user a definitive tactile feedback by way of a sudden reduction in force (from the firing of spring release 136) and allows the handle to displace far past its normal operation longitudinal stroke without resistance, reducing the chance that a user will mistake the post-firing further proximal retraction of spring release 136 or 236 with firing. Both of these factors aid the user in determining successful deployment of distal clip assembly 100.

Referring now to FIGS. 37-39, several different embodiments for the jaw members are described. In FIG. 37, an embodiment of a jaw member 308 is shown. Jaw member 308 is similar to jaw members 108 in that it can be used in the jaw assembly 104 and the distal clip assembly 100. Jaw member 308 also includes a distal end effector similar to distal end effector 120. The main difference between jaw member 308 and jaw member 108 is that jaw member 308 includes a cam slot 322 in a proximal portion 316 of the jaw member 308 where the cam slot 322 includes a locking neck 330 formed by a tapered portion 331, e.g., a linear triangular ramp, where protrusion 131 is more of an arcuate ramp. Once the cam pin (e.g., pin 110) crests the inflection point on the ramp 331, it will snap into place distally in front of the ramp 331, effectively locking the jaw members 308 in a closed position. This geometry allows an easier transmission of axial force to normal force on the internal walls of cam slot 322, requiring less force to initiate locking. The proximal end of triangle locking ramp 331 will also prevent axial transmission of the cam pin after locking is achieved. This locking ramp 331 and its other configurations described in the present disclosure prevent reopening due to the flexible nature of drive-wire 109, which cannot transmit much compressive force to force the jaw members 308 distally to disengage pin, e.g. pin 110, from the portion of cam slot 322 distal of ramp 331.

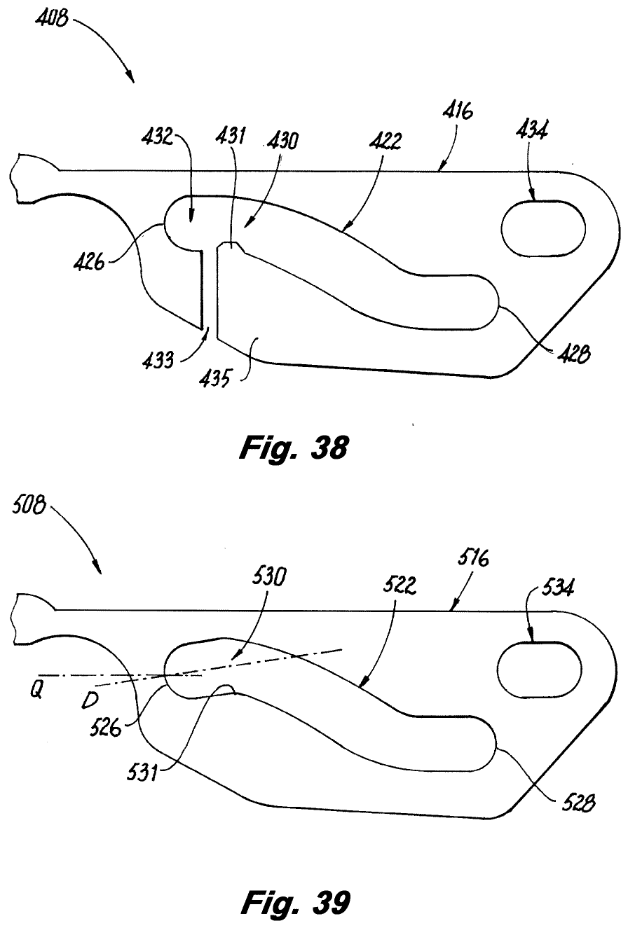

As shown in FIG. 38, another embodiment of a jaw member 408 is shown. Jaw member 408 is similar to jaw members 108 in that it can be used in the jaw assembly 104 and the distal clip assembly 100. Jaw member 408 also includes a distal end effector similar to distal end effector 120. The main difference between jaw member 408 and jaw member 108 is that jaw member 408 includes a cam slot 422 in a proximal portion 416 of the jaw member 408 having a locking neck 430 formed by a protrusion 431. A distal locking area 432, similar to locking area 132, is defined by the locking neck 430. Additionally, the protrusion 431 terminates in a slot 433. Slot 433 is oriented orthogonally to a longitudinal axis Q of the jaw member 108, which when in the closed configuration, is generally parallel to the longitudinal axis A. This open slot 433 creates a cantilever lock arm 435 on the bottom wall of cam slot 422. This results in a decreased force required to lock the clip, and results in a higher rate of successful locking in instances where the jaw members 408 are not perfectly parallel to each other, as deflection in the cantilever lock arm 435 can accommodate some axis offset of the jaw members 408. A distal locking area 432, similar to locking area 132, is positioned distally from the protrusion 431.

As shown in FIG. 39, another embodiment of a jaw member 508 is shown. Jaw member 508 is similar to jaw members 108 in that it can be used in the jaw assembly 104 and the distal clip assembly 100. Jaw member 508 also includes a distal end effector similar to distal end effector 120. The main difference between jaw member 508 and jaw member 108 is that jaw member 508 includes a cam slot 522 in a proximal portion 516 of the jaw member 508 having a locking neck 530 formed by a slope reversal on a distal portion 528 of the cam slot 522. In other words, instead of a distal axis D of distal portion 528 being parallel to a longitudinal axis A of a catheter body, e.g., catheter body 105, distal axis D is angled radially outward relative to axis A resulting in a locking force due to cantilever deflection. In this instance, the user will feel a gradual increase in feedback force, and then a sudden decrease. Once a cam pin, e.g., second pin 110, has crested an inflection point 531 of the pin track (again, relative to the longitudinal axis of the clip body, which is parallel to longitudinal axis A of catheter body at rest) the slope direction changes and begins to force the clip open ever so slightly (0-10 degrees of angulation between jaws. Subsequent unlocking of the jaw members 508 would require equal proximal movement of the cam pin relative to a pivot pin, e.g., second pin 110, which is prevented by the spring force required to pass the cam pin over the inflection point during distal translation. Again, an elongate drive wire, e.g., drive wire 109, will not be able to transmit sufficient compressive force to actuate the cam pin distally, effectively locking the clip. The cam slot 522 of jaw member 508 has the as the added benefit of accommodating some amount of tissue thickness between the jaw members 508 without incurring bending stress in the jaw members 508. A distal locking area 532, similar to locking area 132, is defined by the locking neck 530. A distal locking area 532, similar to locking area 132, is positioned distally from inflection point 531.

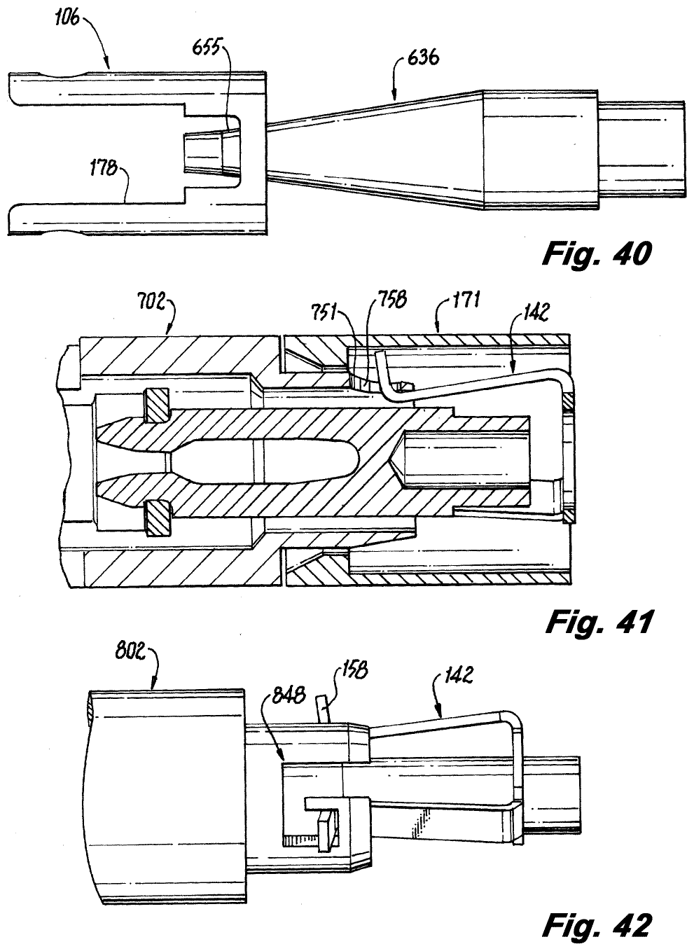

As shown in FIG. 40, alternate configurations of a release pin 636 is shown. Release pin 636 can be used in conjunction with device 10 and yoke 106 in lieu of release pin 136. Release pin 636 includes tapered flat surfaces 655 which allow for misalignment between the proximal receiving portion 133 of the jaw adapter yoke 106, increasing the flexibility in the distal end of the device 10. By tapering flat surfaces 655, the release pin is able to flex in the plane orthogonal to a plane defined by inner facing surface 178 of yoke 106.

As shown in FIG. 41, another embodiment of a distal clip housing 702 can be used in device 10 in lieu of distal clip housing 102. Distal clip housing 702 is generally the same as distal clip housing 102, except that a proximal end 751 of distal clip housing 702 (including the surface containing circumferentially spaced apart apertures 758) is tapered radially inwardly from the distal direction to the proximal direction. This allows for misalignment between distal clip housing and the retention cap 171, increasing the flexibility of the device 10.

As shown in FIG. 42, another embodiment of a distal clip housing 802 can be used in device 10 in lieu of distal clip housing 102. Distal clip housing 802 is generally the same as distal clip housing 102, except that apertures in the distal clip housing 802 are formed as u-shaped slots 848, instead of circumferentially spaced apart apertures 148, to engage with outward extending flanges 158 of shaft spring 142. This allows for assembly via linearly pushing shaft spring 142 and rotating shaft spring 142 until outward extending flanges 158 of shaft spring 142 seat in U-shaped slots 848. This design eliminates the need for radial assembly motion and deflection, vastly simplifying the manufacturing process.

The methods and systems of the present disclosure, as described above and shown in the drawings, provide for a surgical device with superior properties including simplified user feedback, reduced accidental deployment of the clip assembly and a shorter clip body. Additionally, the firing mechanism is elastic, and permanent deformation, e.g., breakage, is not required to deploy the clip assembly. While the apparatus and methods of the subject disclosure have been showing and described with reference to embodiments, those skilled in 10 the art will readily appreciate that changes and/or modifications may be made thereto without departing from the spirit and score of the subject disclosure.

What is claimed is:

1. A device for applying a hemostatic clip assembly, comprising:
a proximal delivery catheter including a proximal handle assembly, an elongated catheter body defining a longitudinal axis and extending distally from the proximal handle assembly, a drive wire movably positioned within the elongated catheter body, a spring release coupled to a distal end of the drive wire, and a shaft spring positioned radially outward from the spring release, wherein the shaft spring includes an annular portion, wherein the spring release is configured and adapted to abut the annular portion of the shaft spring upon proximal translation of the spring release; and
a distal clip assembly removably connected to the distal end of the elongated catheter body, wherein the proximal delivery catheter is configured and adapted to transmit linear motion along the longitudinal axis and torsion about the longitudinal axis to at least a portion of the distal clip assembly.

2. The device as recited in claim 1, wherein the shaft spring is positioned radially inward from the elongated catheter body.

3. The device as recited in claim 1, wherein the annular portion of the shaft spring is positioned around the drive wire, wherein a proximal facing surface of the spring release is configured and adapted to interfere with the annular portion of the shaft spring upon proximal translation of the spring release.

4. The device as recited in claim 1, wherein the shaft spring is configured and adapted to translate proximally relative to the elongated catheter body.

5. The device as recited in claim 1, wherein the distal clip assembly includes a distal clip housing, wherein the shaft spring includes at least one arm removably coupled to the distal clip housing.

6. The device as recited in claim 5, wherein the at least one arm includes an outwardly extending flange that removably engages with an aperture defined in a proximal end of the distal clip housing.

7. The device as recited in claim 6, wherein the outwardly extending flange of the at least one arm is configured and adapted to bend and release from the aperture of the distal clip housing as the spring release moves proximally to move the shaft spring proximally relative to the distal clip housing.

8. The device as recited in claim 1, wherein the distal clip assembly includes a distal clip housing, a jaw adapter yoke slidably positioned within the distal clip assembly, and a jaw assembly having a pair of cooperating jaw members fixed to the jaw adapter yoke by a first pin, the first pin oriented orthogonally relative to the longitudinal axis, wherein at least one of the jaw members is configured and adapted to rotate about the first pin and to rotate about the longitudinal axis.

9. The device as recited in claim 8, wherein the distal clip assembly includes a second pin connecting between the jaw members and the distal clip housing, wherein each jaw member includes a proximal body portion and a distal end effector, wherein the proximal body portion of each jaw member includes a respective cam slot configured and adapted to receive the second pin and a pivot aperture configured and adapted to receive the first pin.

10. The device as recited in claim 9, wherein the cam slots are configured and adapted to translate along the second pin to move axially relative to the distal clip housing and to move the jaw members between a open configuration, where respective distal tips of the jaw members are moved away from one another, a closed configuration where the respective distal tips of the jaw members are approximated towards one another to grasp tissue, and a locked configuration.

11. The device as recited in claim 10, wherein each cam slot includes a distal locking neck projecting into the cam slot defining a distal locking area, wherein the jaw members are in the locked configuration when the second pin is distal relative to the distal locking neck in the distal locking area.

12. The device as recited in claim 8, wherein the jaw adapter yoke includes a proximal receiving portion and the spring release includes a distal portion configured and adapted to be received within the proximal receiving portion of the jaw adapter yoke to transmit axial and rotational force from the drive wire to the jaw adapter yoke.

13. The device as recited in claim 12, wherein the drive wire is coupled to a proximal portion of the spring release to transmit linear and rotational motion from the drive wire to the jaw adapter yoke.

14. The device as recited in claim 13, wherein the distal portion of the spring release is divided into at least two prongs, wherein each prong has a mating surface selectively engageable with an inner surface of the receiving portion of the jaw adapter yoke.

15. The device as recited in claim 14, wherein each prong is configured and adapted to deflect inwardly and release from the receiving portion when an axial force in a proximal direction is applied to the spring release.

16. The device as recited in claim 8, wherein the jaw adapter yoke includes a pair of axially extending spaced apart arms, wherein each arm includes an elongated opening, wherein the first pin is slidably received within each elongated opening, wherein the elongated opening is configured and adapted to allow a first of the jaw members to be angled at a first angle relative to the longitudinal axis and a second of the jaw members to be angled at a second angle relative to the longitudinal axis.

17. The device as recited in claim 8, wherein the spring release includes a distal portion, a proximal portion, and a neck portion therebetween, wherein the distal portion of the spring release is configured and adapted to be received within a bore of the jaw adapter yoke to transmit axial and rotational force from the drive wire to the jaw adapter yoke.

18. A device for applying a hemostatic clip assembly, the device comprising:
a proximal delivery catheter including a proximal handle assembly, an elongated catheter body extending distally from the proximal handle assembly, the elongated catheter body defining a longitudinal axis, a drive wire movably positioned within the elongated catheter body, a spring release coupled to a distal end of the drive wire, and a shaft spring positioned radially outward from the spring release; and
a distal clip assembly removably connected to a distal end of the elongated catheter body, the distal clip assembly including a distal clip housing having a distally facing retaining surface, a jaw adapter yoke slidably positioned within the distal clip assembly, and a jaw assembly having a pair of cooperating jaw members fixed to the jaw adapter yoke, wherein the proximal delivery catheter is configured and adapted to transmit linear motion along the longitudinal axis and torsion about the longitudinal axis to at least a portion of the distal clip assembly, wherein the shaft spring includes at least one arm removably coupled to the distally facing retaining surface of the distal clip housing.

19. The device as recited in claim 18, wherein the jaw adapter yoke includes a pair of axially extending spaced apart arms, wherein each arm includes an elongated opening, wherein a first pin is slidably received within each elongated opening, wherein the elongated opening is configured and adapted to allow a first of the jaw members to be angled at a first angle relative to the longitudinal axis and a second of the jaw members to be angled at a second angle relative to the longitudinal axis.

20. The device as recited in claim 18, wherein the shaft spring is positioned radially inward from the elongated catheter body.

21. The device as recited in claim 18, wherein the shaft spring includes an annular portion positioned around the drive wire, wherein a proximal facing surface of the spring release is configured and adapted to abut the annular portion of the shaft spring upon proximal translation of the spring release.

22. The device as recited in claim 18, wherein the pair of cooperating jaw members are fixed to the jaw adapter yoke by a first pin, the first pin oriented orthogonally relative to the longitudinal axis.

23. The device as recited in claim 22, wherein the distal clip assembly includes a second pin connecting between the jaw members and the distal clip housing, wherein each jaw member includes a proximal body portion and a distal end effector, wherein the proximal body portion of each jaw member includes a respective cam slot configured and adapted to receive the second pin and a pivot aperture configured and adapted to receive the first pin.

24. The device as recited in claim 23, wherein the cam slots are configured and adapted to translate along the second pin to move axially relative to the distal clip housing and to move the jaw members between an open configuration, where respective distal tips of the jaw members are moved away from one another, a closed configuration, where the respective distal tips of the jaw members are approximated towards one another to grasp tissue, and a locked configuration.

25. The device as recited in claim 24, wherein each cam slot includes a distal locking neck projecting into the cam slot defining a distal locking area, wherein the jaw members are in the locked configuration when the second pin is distal relative to the distal locking neck in the distal locking area.

26. The device as recited in claim 18, wherein the proximal delivery catheter includes a drive wire movably positioned within the elongated catheter body, wherein a distal end of the drive wire is coupled to a proximal portion of the spring release to transmit linear and rotational motion from the drive wire to the jaw adapter yoke.

27. The device as recited in claim 26, wherein the shaft spring includes an annular portion, wherein the spring release is configured and adapted to abut the annular portion of the shaft spring upon proximal translation of the spring release.

28. The device as recited in claim 18, wherein the at least one arm includes an outwardly extending flange that removably engages with the distally facing retaining surface of an aperture defined in a proximal end of the distal clip housing.

29. The device as recited in claim 28, wherein the outwardly extending flange of the at least one arm is configured and adapted to bend and release from the aperture of the distal clip housing as the spring release moves proximally to move the shaft spring proximally relative to the distal clip housing.

30. The device as recited in claim 18, wherein the spring release includes a distal portion, a proximal portion, and a neck portion therebetween, wherein the distal portion of the spring release is configured and adapted to be received within a bore of the jaw adapter yoke to transmit axial and rotational force from the drive wire to the jaw adapter yoke.

31. The device as recited in claim 18, wherein the jaw adapter yoke includes a proximal receiving portion, and wherein the spring release includes a distal portion configured and adapted to be received within the proximal receiving portion of the jaw adapter yoke to transmit axial and rotational force from the drive wire to the jaw adapter yoke.

32. The device as recited in claim 31, wherein the distal portion of the spring release is divided into at least two prongs, wherein each prong has a mating surface selectively engageable with an inner surface of the receiving portion of the jaw adapter yoke.

33. The device as recited in claim 32, wherein each prong is configured and adapted to deflect inwardly and release from the receiving portion when an axial force in a proximal direction is applied to the spring release.

34. A device for applying a hemostatic clip assembly, the device comprising:
a proximal delivery catheter including a proximal handle assembly, an elongated catheter body extending distally from the proximal handle assembly, the elongated catheter body defining a longitudinal axis; and
a distal clip assembly removably connected to a distal end of the elongated catheter body, the distal clip assembly including a distal clip housing having a distally facing retaining surface, a jaw adapter yoke slidably positioned within the distal clip assembly, and a jaw assembly having a pair of cooperating jaw members fixed to the jaw adapter yoke with a first pin, wherein the proximal delivery catheter is configured and adapted to transmit linear motion along the longitudinal axis and torsion about the longitudinal axis to at least a portion of the distal clip assembly, wherein the jaw adapter yoke includes a pair of axially extending spaced apart arms, wherein each arm includes an elongated opening, wherein the first pin is slidably received within each elongated opening, wherein the elongated opening is configured and adapted to allow a first of the jaw members to be angled at a first angle relative to the longitudinal axis and a second of the jaw members to be angled at a second angle relative to the longitudinal axis.

35. The device as recited in claim 34, wherein the proximal delivery catheter includes a drive wire movably positioned within the elongated catheter body, a spring release coupled to a distal end of the drive wire, and a shaft spring positioned radially outward from the spring release.

36. The device as recited in claim 35, wherein the shaft spring includes at least one arm removably coupled to the distally facing retaining surface of the distal clip housing.

37. The device as recited in claim 36, wherein the at least one arm includes an outwardly extending flange that removably engages with the distally facing retaining surface of an aperture defined in a proximal end of the distal clip housing.

38. The device as recited in claim 37, wherein the outwardly extending flange of the at least one arm is configured and adapted to bend and release from the aperture of the distal clip housing as the spring release moves proximally to move the shaft spring proximally relative to the distal clip housing.

39. The device as recited in claim 35, wherein a distal end of the drive wire is coupled to a proximal portion of the spring release to transmit linear and rotational motion from the drive wire to the jaw adapter yoke.

40. The device as recited in claim 35, wherein the shaft spring includes an annular portion, wherein the spring release is configured and adapted to abut the annular portion of the shaft spring upon proximal translation of the spring release.

41. The device as recited in claim 35, wherein the spring release includes a distal portion, a proximal portion, and a neck portion therebetween, wherein the distal portion of the spring release is configured and adapted to be received within a bore of the jaw adapter yoke to transmit axial and rotational force from the drive wire to the jaw adapter yoke.

42. The device as recited in claim 35, wherein the shaft spring is positioned radially inward from the elongated catheter body.

43. The device as recited in claim 35, wherein the shaft spring includes an annular portion positioned around the drive wire, wherein a proximal facing surface of the spring release is configured and adapted to abut the annular portion of the shaft spring upon proximal translation of the spring release.

44. The device as recited in claim 34, wherein the distal clip assembly includes a second pin connecting between the jaw members and the distal clip housing, wherein each jaw member includes a proximal body portion and a distal end effector, wherein the proximal body portion of each jaw member includes a respective cam slot configured and adapted to receive the second pin and a pivot aperture configured and adapted to receive the first pin.

45. The device as recited in claim 44, wherein the cam slots are configured and adapted to translate along the second pin to move axially relative to the distal clip housing and to move the jaw members between an open configuration, where respective distal tips of the jaw members are moved away from one another, a closed configuration, where the respective distal tips of the jaw members are approximated towards one another to grasp tissue, and a locked configuration.

46. The device as recited in claim 44, wherein each cam slot includes a distal locking neck projecting into the cam slot defining a distal locking area, wherein the jaw members are in the locked configuration when the second pin is distal relative to the distal locking neck in the distal locking area.

47. The device as recited in claim 35, wherein the jaw adapter yoke includes a proximal receiving portion, and wherein the spring release includes a distal portion configured and adapted to be received within the proximal receiving portion of the jaw adapter yoke to transmit axial and rotational force from the drive wire to the jaw adapter yoke.

48. The device as recited in claim 47, wherein the distal portion of the spring release is divided into at least two prongs, wherein each prong has a mating surface selectively engageable with an inner surface of the receiving portion of the jaw adapter yoke.

49. The device as recited in claim 48, wherein each prong is configured and adapted to deflect inwardly and release from the receiving portion when an axial force in a proximal direction is applied to the spring release.

50. The device as recited in claim 34, wherein the first angle is different from the second angle.

51. A hemostatic clip assembly, the assembly comprising:
a distal clip housing defining a longitudinal axis;
a jaw adapter yoke slidably positioned within the distal clip housing;
a jaw assembly having a pair of cooperating jaw members fixed to the jaw adapter yoke by a first pin, the first pin oriented orthogonally relative to the longitudinal axis, wherein the jaw adapter yoke is configured and adapted to translate axially along the longitudinal axis and rotate about the longitudinal axis, wherein at least one of the jaw members is configured and adapted to rotate about the first pin and to rotate about the longitudinal axis, wherein the jaw adapter yoke includes a pair of axially extending spaced apart arms, wherein each arm includes an elongated opening, wherein the first pin is slidably received within each elongated opening, wherein the elongated opening is configured and adapted to allow a first of the jaw members to be angled at a first angle relative to the longitudinal axis and a second of the jaw members to be angled at a second angle relative to the longitudinal axis

52. The hemostatic clip assembly as recited in claim 51, further comprising a second pin connecting between the jaw members and the distal clip housing, wherein each jaw member includes a proximal body portion and a distal end effector, wherein the proximal body portion of each jaw member includes a respective cam slot configured and adapted to receive the second pin and a pivot aperture configured and adapted to receive the first pin.

53. The hemostatic clip assembly as recited in claim 52, wherein the cam slots are configured and adapted to translate along the second pin to move axially relative to the distal clip housing and to move the jaw members between the open configuration where respective distal tips of the jaw members are moved away from one another, the closed configuration where the respective distal tips of the jaw members are approximated towards one another to grasp tissue, and a locked configuration.

54. The hemostatic clip assembly as recited in claim 51, wherein the jaw adapter yoke includes a proximal receiving portion configured and adapted receive a spring release of a proximal delivery catheter.

55. A method for firing a hemostatic clip assembly the method comprising:
positioning a distal clip assembly proximate to a target location, wherein the distal clip assembly includes a distal clip housing, a jaw adapter yoke slidably positioned within the distal clip assembly, and a jaw assembly having a pair of cooperating jaw members fixed to the jaw adapter yoke by a first pin; and
translating an actuation portion of a proximal handle assembly of a proximal delivery catheter relative to a grasping portion of the proximal handle assembly in at least one of a proximal direction or a distal direction, wherein the proximal delivery catheter includes an elongated catheter body extending distally from the proximal handle assembly, the elongated catheter body defining a longitudinal axis, the actuation portion operatively connected to the jaw adapter yoke via a drive wire and a spring release to transmit linear motion along the longitudinal axis and torsion about the longitudinal axis to the jaw adapter yoke, wherein the linear motion of the jaw adapter yoke transmits a linear component of motion to at least one jaw member and a cam slot of at least one jaw member to translate the cam slot along a second pin connecting between at least one of the jaw members and the distal clip housing, thereby rotating at least one of the jaw members about the first pin and to rotate about the longitudinal axis, wherein translating the actuation portion includes translating the spring release in the proximal direction causing abutting between the spring release and an annular portion of the shaft spring, wherein the shaft spring is coupled to a proximal end of the distal clip housing via an outwardly extending flange of at least one arm extending from the annular portion of the shaft spring, wherein the abutting causes the outwardly extending flange to bend inward and disengage from the proximal end of the distal clip housing.

56. The method as recited in claim 55, wherein translating the actuation portion includes translating the actuation portion further in the proximal direction to transmit further linear motion in the proximal direction to the spring release, the further linear motion in a proximal direction de-coupling a distal portion of the spring release from a receiving portion of the jaw adapter yoke.
